(19) United States
(12) Patent Application Publication
(10) Pub. No.: US 2011/0053777 A1
(43) Pub. Date: Mar. 3, 2011

(54) Resistance to Acetolactate Synthase-Inhibiting Herbicides

(76) Inventors: James H. Oard, Baton Rouge, LA (US); Nengyi Zhang, Baton Rouge, LA (US); Dearl E. Sanders, Clinton, LA (US)

(21) Appl. No.: 12/303,888

(22) PCT Filed: Jun. 15, 2006
(86) PCT No.: PCT/US06/23247

§ 371 Date: Nov. 13, 2009

(51) Int. Cl. A01N 43/52; C07H 21/04; C12N 15/63; C12N 5/10; A01H 5/00; A01H 1/00; A01N 47/34; A01P 13/00

(52) U.S. Cl. 504/276; 536/23.2; 435/320.1; 435/419; 800/295; 800/278; 536/23.1; 504/329

(57) ABSTRACT

Nucleotide sequences are disclosed that may be used to impart herbicide resistance to green plants. The sources of novel herbicide resistance were originally isolated in mutant Coreopsis plants. Green plants transformed with these sequences are resistant to herbicides that normally inhibit acetolactate synthase (ALS), particularly imidazolinone and sulfonylurea herbicides.

### TECHNICAL FIELD

This invention pertains to herbicide resistant plants, and to nucleotide sequences conferring herbicide resistance to plants, particularly resistance to herbicides that normally interfere with the plant enzyme acetolactate synthase (ALS), such herbicides including for example those of the imidazolinone class and those of the sulfonylurea class.

### BACKGROUND ART

The development of novel herbicide resistance in plants offers significant production and economic advantages. Weeds are a constant limitation to optimal commercial crop production and cause substantial yield losses in all growing areas of the world. The use of herbicides is an effective, easy, and comparatively inexpensive approach to control weeds. Acetolactate synthase (ALS) (also known as acetohydroxyacid synthase, AHAS; E.C. 4.1.3.18), which catalyses the first common step in the biosynthesis of the branched-chain amino acids in plants, is a target of several herbicide groups, including sulfonylureas, imidazolinones, triazolopyrimidines, pyrimidinyl oxybenzoates, and sulfonylaminocarbonyl-triazolinones. These herbicides block the biosynthesis of the essential amino acids valine, leucine, and isoleucine. It is believed that ALS-inhibiting herbicides cause plant death essentially by starving the plants for these amino acids. Secondary effects of ALS inhibition may also play a role, including buildup of α-ketobutyrate, disruption of protein synthesis, and disruption of photosynthate transport.

The mature ALS protein has approximately 670 amino acids, and its sequence is highly conserved across species. Most diploid plant species that have been studied have a single ALS locus (e.g., Arabidopsis thaliana and Xanthium sp.), with some exceptions. Maize (Zea mays) has two ALS loci, and sunflower (Helianthus annuus) three. Tetraploid tobacco (Nicotiana tabacum) has two loci; Brassica species possess five loci; and Gossypium napus contains six loci. Coreopsis tinctoria is diploid, but the number of ALS loci in the C. tinctoria genome is currently unknown.

Advantages of herbicides that inhibit ALS, such as the sulfonylureas, imidazolinones, and others, include their high efficacy, broad-spectrum weed control, low application rates, and relative environmental safety. Herbicidally-effective sulfonylureas include chlorsulfuron (Glean®) and sulfometuron methyl (Oust®). Herbicidally-effective imidazolinones include imazethapyr (Pursuit®), imazaquin (Scepter®), imazapic (Plateau®), and imazapyr (Arsenal®). Worldwide, there are over thirty commercial ALS-inhibiting herbicides, indicating their importance for weed management in a wide range of crops.

Resistance to ALS-inhibiting herbicides has been reported in several plant species, both monocots and dicots. It is difficult to predict in advance where herbicide resistance will arise. When herbicide resistance does arise, the mere observation of resistance does not, in itself, indicate what the mechanism of resistance is. The mechanism can usually only be determined by laboratory testing. In some cases, resistance has been attributed to an altered form of the ALS enzyme that is less sensitive to certain herbicides. In other cases, resistance has been attributed to enhanced herbicide metabolism, rapid detoxification of the herbicide. In still other cases, the mechanism of resistance remains unknown.

Further, different point mutations in different locations in the ALS gene can result in differing herbicide resistance profiles, both within a class of ALS-inhibiting herbicides, and between different classes of ALS-inhibiting herbicides.

There have been no prior reports describing the effect of compounding different point mutations within a single ALS allele. If point mutations x and y in the ALS gene are both associated with ALS-inhibiting herbicide resistance, it is unknown whether combining both point mutations x and y in a single gene will result in the same level of resistance, or different levels of resistance. Some mutations may be effectively “redundant” with one another, meaning that there is no substantial cumulative effect from having both mutations in the same gene. Some mutations may have a synergistic effect, so there is an enhanced effect. Some mutations may affect different classes of herbicide, so that the combination expands the range of herbicides to which resistance is expressed, but without substantially changing the levels of resistance to any individual herbicide. The effect of some mutations may be to negate one another, or even to reduce the enzyme's overall activity to the point that it is detrimental to the plant. Until there has been further experimental work, the effect of combining two mutations within the same ALS allele is unpredictable, even if the separate effects of the individual mutations are known.

An additional factor that will, in some cases, make it difficult to predict the effect of ALS mutations is that earlier reports have said little about regulation of the ALS gene. It is possible that differences in the regulation of the ALS gene may have a significant effect on herbicide resistance and herbicide sensitivity, although prior reports have not provided much information that would be useful in making predictions. If a previously unknown regulatory sequence were found for an ALS gene in a particular species, then it would be particularly difficult to predict a priori what the expression patterns for ALS will be in that species, and what the susceptibility or resistance of the plant to different ALS-herbicides might be.

Mutations imparting resistance to ALS-inhibiting herbicides have been identified both in mutants generated in the laboratory, e.g., tobacco (Nicotiana tabacum), Arabidopsis thaliana, maize (Zea mays), rice (Oryza sativa), and Brassica napus; and in the field, in both crops and weeds, presumably resulting from selective pressure, e.g., lettuce (Lactuca serriola), kochia (Kochia scoparia), cocklebur (Xanthium strumarium), Raphanus raphanistrum, Lindernia, and Amaranthus blitoides. Most ALS mutant lines, whether from laboratory or field sources, do not show resistance to a broad spectrum of ALS-inhibiting herbicides. There is an unfilled need for new sources of ALS that exhibit a broad spectrum of resistance, and that may be used to transfer high levels of herbicide resistance to commercial crops.

Sulfometuron methyl, a strong inhibitor of ALS, is widely used as a non-crop herbicide. It has limited selectivity and is primarily used as a “bare ground” herbicide. It is used in Louisiana, for example, for the control of unwanted vegetation on bermudagrass roadsides and for herbaceous weed control in pine forests. Sulfometuron methyl does have marginal selectivity for bermudagrass (Cynodon dactylon).

Mutations conferring resistance to imidazolinone herbicides have been reported at amino acid positions (codons) Ala122, Pro197, Ala205, Trp574, and Ser653 in several plant species. (Unless context indicates otherwise, all amino acid positions described in the present specification and claims are numbered by correspondence to the sequence reported for A. thaliana by Sathasivan K., Haughn G. W., and Murai N. 1990. Nucleotide sequence of a mutant acetohydroxyacid synthase gene from an imidazolinone-resistant Arabidopsis thaliana var. Columbia. Nucleic Acids Res. 18:2188; and Sathasivan K., Haughn G. W., and Murai N. 1991. Molecular basis of imidazolinone herbicide resistance in Arabidopsis thaliana var Columbia. Plant Physiol. 97:1044-1050. This numbering convention does not, however, apply to the formal “Sequence Listing” section of this specification; nor to the discussion in paragraphs 0014-0040 below.) Mutations at Ala122, Ser653, and Ala205 have been reported to confer resistance to imidazolinones, but not sulfonylureas. On the other hand, resistance to sulfonylureas has been reported for the mutation Pro197. The Trp574 to Leu mutation has been reported to confer resistance to representatives of all five families of ALS-inhibiting herbicides. Commercial varieties of rice (Oryza sativa), maize (Zea mays), oilseed rape (Brassica napus), sunflower, and wheat that are resistant to imidazolinone herbicides have been released. Clearfield rice varieties CL121 and CL141 were developed by chemical mutagenesis of seeds, with a mutation at Gly654. Seeds from the rice variety Cypress were treated with the chemical EMS to produce a mutation at Ser 653, leading to the variety CL161. Four separate herbicide-resistance mutations have been reported in the corn ALS gene (codons Ala122, Ala155, Trp574, Ser653), produced from either cell culture or chemical mutagenesis. Oilseed rape mutants PM1 and PM2 were derived from microspore mutagenesis, with mutations at Ser653 and Trp574, respectively. Clearfield varieties of sunflower were produced with a mutation at Ala205. Both winter and spring varieties of imidazolinone-resistant wheat have been released, with a mutation at Ser653. Mutations at Ala122, Pro197, and at other, unknown sites have been reported to confer resistance to imidazolinone herbicides in sugarbeet (Beta vulgaris), cotton (Gossypium hirsutum), soybean (Glycine max), lettuce (Lactuca sativa), tomato (Lycopersicon esculentum), and tobacco (Nicotiana tabacum).

At least 23 herbicide-resistant weed species have been identified with mutations in the ALS gene. See http://www.weedscience.org/mutations/MutDisplay.aspx (Tranel 2006, visited on May 27, 2006), which presents a table that is updated by its authors from time to time. Included among the mutations reported in this table are Ala122, Pro197, Ala205, Trp574, and Ser653. See also P. Tranel et al., 2002. Weed Science. 50:700-712.

It has been reported that enzyme-based resistance to ALS herbicides is generally inherited in a semi-dominant manner. Homozygotes typically have much higher resistance to herbicide than control, with heterozygotes having intermediate levels of resistance, but still substantially higher than control. The manner in which resistance is inherited in C. tinctoria has not previously been reported.

A three dimensional molecular model to predict the effect of ALS mutations on resistance to different herbicide classes was recently reported by McCourt J. A., Pang S. S., King-Scott J., Guddat L. W., and Duggleby R. G. 2006. Herbicide-binding sites revealed in the structure of plant acetohydroxyacid synthase. PNAS 103:569-573.

S. Tan et al., Evans R. R., Dahmer M. L., Singh B. K., and Shaner D. L. 2005. Imidazolinone-tolerant crops: history, current status and future. Pest Manag. Sci. 61:246-257 provides a review of the development of several imidazolinone-tolerant crops, and the locations of point mutations for resistance, including Ala122, Pro197, Ala205, Trp574, and Ser653.

U.S. Pat. No. 4,761,373 describes the development of mutant herbicide-resistant maize plants through exposing tissue cultures to herbicide. The mutant maize plants were said to have an altered enzyme, namely acetohydroxyacid synthase, that conferred resistance to certain imidazolinone and sulfonamide herbicides. See also U.S. Pat. Nos. 5,304,732, 5,331,107, 5,718,079, 6,211,438, 6,211,439, and 6,222,100.

Lee et al., “The Molecular Basis of Sulfonylurea Herbicide Resistance in Tobacco,” The EMBO J., vol. 7, no. 5, pp. 1241-1248 (1988), describe the isolation and characterization from Nicotiana tabacum of mutant genes specifying herbicide resistant forms of acetolactate synthase, and the reintroduction of those genes into sensitive lines of tobacco.

Saxena at al., “Herbicide Resistance in Datura innoxia,” Plant Physiol., vol. 86, pp. 863-867 (1988) describe several Datura innoxia lines resistant to sulfonylurea herbicides, some of which were also found to be cross-resistant to imidazolinone herbicides.

U.S. Pat. No. 5,767,366 discloses transformed plants with genetically engineered imidazolinone resistance, conferred through a gene cloned from a plant such as a mutated Arabidopsis thaliana.

Examples of resistance to AHAS-inhibiting herbicides in various plants are disclosed in U.S. Pat. No. 5,013,659; K. Newhouse et al., “Mutations in corn (Zea mays L.) Conferring Resistance to Imidazolinone Herbicides,” Theor. Appl. Genet., vol. 83, pp. 65-70 (1991); K. Sathasivan et al., “Molecular Basis of Imidazolinone Herbicide Resistance in Arabidopsis thaliana var Columbia,” Plant Physiol. vol. 97, pp. 1044-1050 (1991); B. Miki et al., “Transformation of Brassica napus canola cultivars with Arabidopsis thaliana Acetohydroxyacid Synthase Genes and Analysis of Herbicide Resistance,” Theor. Appl. Genet., vol. 80, pp. 449-458 (1990); P. Wiersma at al., “Isolation, Expression and Phylogenetic Inheritance of an Acetolactate Synthase Gene from Brassica napus,” Mol. Gen. Genet., vol. 219, pp. 413-420 (1989); J. Odell et al., “Comparison of Increased Expression of Wild-Type and Herbicide-Resistant Acetolactate Synthase Genes in Transgenic Plants, and Indication of Postranscriptional Limitation on Enzyme Activity,” Plant Physiol., vol. 94, pp. 1647-1654 (1990); published international patent application WO 92/08794; U.S. Pat. No. 5,859,348; published international patent application WO 98/02527; published European Patent Application EP 0 965 265 A2, and published international patent application WO 90/14000.

S. Sebastian at al., “Soybean Mutants with Increased Tolerance for Sulfonylurea Herbicides,” Crop. Sci., vol. 27, pp. 948-952 (1987) discloses soybean mutants resistant to sulfonylurea herbicides. See also U.S. Pat. No. 5,084,082.

ALS herbicide-resistant rice plants, rice-derived sequences encoding that ALS herbicide resistance, and the transformation of other green plants with those rice-derived sequences are disclosed in U.S. Pat. Nos. 6,274,796, 6,943,280, and 7,019,196.

U.S. Pat. No. 4,443,971 discloses a method for preparing herbicide tolerant plants by tissue culture in the presence of herbicide. U.S. Pat. No. 4,774,381 discloses sulfonylurea (sulfonamide) herbicide-resistant tobacco plants prepared in such a manner.

U.S. Pat. No. 5,773,702 discloses sugar beets with a resistant mutant AHAS enzyme, derived from cell cultures grown in the presence of herbicide.

U.S. Pat. No. 5,633,437 discloses a herbicide resistant AHAS enzyme and gene isolated from cockleburs.

U.S. Pat. No. 5,767,361 discloses a mutant, resistant AHAS enzyme from maize. The definitions of the U.S. Pat. No. 5,767,361 patent are incorporated into the present disclosure by reference, to the extent that those definitions are not inconsistent with the present disclosure, as are that patent's descriptions of certain genetic transformation techniques for plants. See also U.S. Pat. No. 5,731,180.

U.S. Pat. No. 5,605,011 discloses resistant acetolactate synthase enzymes derived from callus culture of tobacco cells in the presence of herbicide, from spontaneous mutations of the ALS gene in yeast; EMS-induced mutations in Arabidopsis seeds; certain modifications of those enzymes; and the transformation of various plants with genes encoding the resistant enzymes. These patents disclose several techniques for modifying AHAS genes to produce herbicide-resistant AHAS enzymes, and for transforming plants with those genes.

U.S. Pat. Re 35,661 discloses lettuce plants with enhanced resistance to herbicides that target the enzyme acetolactate synthase. The initial source of herbicide resistance was a prickly lettuce weed infestation in a grower's field, an infestation that was not controlled with commercial sulfonylurea herbicides.

Following are selected data taken from various references concerning the locations of certain imidazolinone or sulfonylurea herbicide tolerance mutations in AHAS/ALS from various species. No attempt has been made to reconcile or align the different nucleotide or amino acid numbering systems used in the different references discussed below. Contrary to the general convention followed in this specification, in the following paragraphs 0030-0040, the sequence numbering systems are those used by the respective authors.

Lee et al. (1988) reported that there were two homologous ALS genes in Nicotiana tabacum. The sulfonylurea herbicide-resistant C3 mutant in one ALS gene had a Pro-Gln replacement at amino acid 196; while the sulfonylurea herbicide-resistant S4-Hra mutant in the other ALS gene had two amino acid changes: Pro-Ala at amino acid 196, and Trp-Leu at amino acid 573.

Sathasivan et al. (1990), Sathasivan et al. (1991), and U.S. Pat. No. 5,767,366 reported a G-A nucleotide substitution at position 1958, corresponding to a Ser-Asn substitution at position 653, in an imidazolinone herbicide-resistant Arabidopsis thaliana.

Wiersma et al. (1989) reported sulfonylurea herbicide resistance in tobacco plants that had been transformed with a mutant Brassica napus ALS gene, in which codon 173 had been altered by site-directed mutagenesis to replace Pro with Ser.

European patent application 0 257 993 A2 reported several spontaneous mutations in the yeast (Saccharomyces cerevisiae) ALS gene that resulted in sulfonylurea herbicide resistance: at amino acid position 121, a substitution of wild-type Gly by Ser; at amino acid position 122, a substitution of wild-type Ala by Pro, Asp, Val, or Thr; at position 197, a substitution of wild-type Pro by Ser or Arg; at position 205, a substitution of wild-type Ala by Asp or Thr; at position 256, a substitution of wild-type Lys by Glu, Thr, or Asn; at position 359, a substitution of wild-type Met by Val; at position 384, a substitution of wild-type Asp by Glu, Val, or Asn; at position 588, a substitution of wild-type Val by Ala; at position 591, a substitution of wild-type Trp by Arg, Cys, Gly, Leu, Ser, or Ala; at position 595, a substitution of wild-type Phe by Leu. The same patent application reported several site-directed mutations of the yeast ALS gene at some of the same positions to also produce sulfonylurea herbicide resistance: at amino acid position 122, a substitution of wild-type Ala by Ser, Val, Thr, Pro, Asn, Ile, His, Arg, Leu, Tyr, Cys, Phe, Glu, Met, Lys, Gln, or Trp; at position 205, a substitution of wild-type Ala by Arg, Cys, Glu, or Trp; at position 256, a substitution of wild-type Lys by Asp, Gly, Leu, Pro, or Trp; at position 359, a substitution of wild-type Met by Pro or Glu; at position 384, a substitution of wild-type Asp by Pro, Trp, Ser, Gly, Cys, or Lys; at position 591, a substitution of wild-type Trp by Asp, Glu, Phe, His, Tyr, Ile, Val, Lys, Arg, Met, Asn, Gln, or Thr. See also U.S. Pat. No. 5,605,011, which also describes experimental data for the following site-directed mutations: at amino acid 121, a substitution of wild-type Gly by Asn or Ala; at amino acid 197, a substitution of wild-type Pro by Gln, Glu, Ala, Gly, Trp, Tyr, Cys, or Val; at amino acid 205, a substitution of wild-type Ala by Tyr, Val, or Asn; at amino acid 359, a substitution of wild-type Met by Gln, Lys, Tyr, or Cys; at position 583, a substitution of wild-type Val by Ser, Asn, Trp, or Cys; and at position 595, a substitution of wild-type Phe by Gly, Asn, Arg, Cys, Pro, Ser, or Trp. Other amino substitutions at the same positions are also described prophetically, without experimental data. See also U.S. Pat. No. 5,013,659.

WO 98/02527 reported sulfonylurea and triazolopyrimidine resistance in a line of sugar beets resulting from a C-T substitution at nucleotide 562, corresponding to a Pro-Ser substitution at amino acid 188. This same reference also reported sulfonylurea, imidazolinone, and triazolopyrimidine resistance in a second line of sugar beets resulting from two mutations: The same mutation as reported in the first line (from which the second line had been derived), coupled with a G-A substitution at nucleotide 337, corresponding to an Ala-Thr substitution at amino acid 113. See also WO 98/02526, U.S. Pat. Nos. 5,859,348 and 5,773,702.

WO 96/33270 describes a number of designed or predicted mutations from a structure-based modeling method, that were said to induce imidazolinone tolerance in AHAS Experimental results confirming such tolerance in mutated Arabidopsis AHAS, either in vitro or in transformed tobacco plants in vivo, were provided for the following substitutions: Met-Ile at amino acid position 124, Met-His at position 124, Arg-Glu at position 199, and Arg-Ala at position 199. See also U.S. Pat. Nos. 5,928,937 and 5,853,973.

WO 92/08794 reported imidazolinone resistance in two lines of maize. One had a G-A substitution at nucleotide position 171, resulting in an Ala-Thr substitution at the corresponding amino acid position. The other had a G-A substitution at position 1888, resulting in a Ser-Asn substitution at the corresponding amino acid position.

U.S. Pat. No. 5,731,180 reported imidazolinone resistance in maize resulting from a G-A substitution at nucleotide position 1898, resulting in a Ser-Asn substitution at amino acid position 621. See also U.S. Pat. No. 5,767,361 and European patent application 0 525 384.

U.S. Pat. No. 5,633,437 reported imidazolinone resistance in cockleburs, characterized by five differences between resistant ALS enzyme biotypes and sensitive biotypes: Lys-Glu at amino acid position 63, Phe-Leu at position 258, Gln-His at position 269, Asn-Ser at position 522, and Trp-Leu at position 552. The changes at positions 522 and 552 were thought to be particularly important.

T. Shimizu et al., “Oryza sativa ALS mRNA for acetolactate synthase, complete cds, herbicide resistant biotype,” BLAST accession number AB049823 (April 2001) reported a nucleotide sequence and inferred amino acid sequence for a rice ALS that was said to be herbicide resistant, although the nature of the herbicide resistance was not specified in the BLAST entry. As compared to a contemporaneous wild type ALS for the same rice variety (Kinmaze), the inferred amino acid sequence for the resistant ALS appeared to display two differences: a Trp-Leu substitution at position 548, and a Ser-Ile substitution at position 627.

U.S. Pat. No. 6,943,280 reports herbicide-resistant AHAS sequences in rice with, inter alia, a serine-to-asparagine mutation at amino acid 627; or a serine-to-lysine mutation at amino acid 627 coupled with a frame-shift mutation leading to a stop codon soon thereafter.

Resistance to ALS-inhibiting herbicides has been observed in a line of Coreopsis tinctoria for several years. The resistant Coreopsis were first observed growing along a highway in Louisiana where sulfonylurea herbicide had been sprayed. This herbicide-resistant Coreopsis has been in public use, for example along Louisiana highways, more than one year before the priority date of this patent application. However, there have been no prior reports concerning the underlying mechanism for herbicide-resistance in Coreopsis. It has not previously been reported whether the resistance is due to a resistant ALS enzyme, enhanced metabolic breakdown of the enzyme, altered regulation of the ALS gene, or some different mechanism entirely.

Coreopsis sp. are annual flowering plants in the Compositae, Asteraceae, or sunflower family. There are both domesticated and wild strains within the genus. Domesticated strains are widely grown, either as bedding plants or from seed in landscapes. Wild strains are found in the United States throughout the South and Midwest. Coreopsis tinctoria is often used as a roadside wildflower in the South. Coreopsis tinctoria seeds are produced commercially and marketed for the roadside beautification.

A resistant population of Coreopsis tinctoria was serendipitously discovered thriving alongside a Louisiana highway near Chase, La. in an area where it should not have been growing at all, as the area had been sprayed multiple times with the herbicide sulfometuron methyl (trade name Oust™) to control roadside weeds. Personnel from the Louisiana Department of Transportation and Development (DOTD) were the first to observe that certain Coreopsis at this site appeared to be unaffected by the herbicide. More than one year before the priority date of the present application, DOTD had placed the herbicide-resistant Coreopsis seeds and plants in public use by planting them for roadside beautification alongside several public highways in Louisiana, in areas where ALS-inhibiting herbicides were used for weed control. See J. LaBorde et al., “Characterization of flower types found in a selected population of plains Coreopsis,” poster presented at American Society of Horticultural Science meeting (Mobile, Ala. 2004); and D. Sanders, “Looking for herbicide resistant wildflowers,” talk presented at National Roadside Vegetation Management Association (Point Clear Ala., 2003).

### SUMMARY OF THE INVENTION

We have discovered a unique, herbicide resistant, acetolactate synthase mutant allele, originally derived from Coreopsis tinctoria. The novel allele expresses resistance to a broad range of ALS-resistant herbicides, and is resistant to very high levels of herbicide. The novel allele has a novel combination of point mutations in its coding sequence that contributes to these unique herbicide-resistance properties. The Coreopsis tinctoria ALS gene is under the control of a novel promoter that has not previously been reported for other ALS genes.

Coreopsis tinctoria mutants from the field showed high levels of resistance at the whole-plant level to both sulfonylurea and imidazolinone herbicides.

We conducted ALS enzyme assays on both resistant and susceptible C. tinctoria, using sulfometuron methyl, chlorsulfuron, and imazapyr as ALS inhibitors. The results indicated that the ALS enzyme in the C. tinctoria mutants either contributed to or was completely responsible for the observed herbicide resistance phenotype. Without wishing to be bound by this hypothesis, it may be the case that the novel promoter also provides a contribution to the overall level of herbicide resistance. However, it is the case that the mutant ALS enzyme itself is highly resistant, even if no resistance were attributable to the promoter.

The novel allele has a novel combination of two point mutations in the ALS coding sequence that together impart pre-emergence resistance, post-emergence resistance, or both pre-emergence resistance and post-emergence resistance to a broad range of herbicides. Resistance has been demonstrated to date against at least the following herbicides: sulfometuron methyl (Oust®), imazapyr (Arsenal®), metsulfuron methyl (Escort®), sulfosulfuron (Outrider®), imazapic (Plateau®), flazasulfuron (Katana®), and triasulfuron (Amber®). With further testing, resistance to most if not all of the following herbicides is expected to be demonstrated also: imazethapyr, imazamox, imazaquin, chlorimuron ethyl, rimsulfuron, thifensulfuron methyl, pyrithiobac sodium, tribenuron methyl, and nicosulfuron; as well as additional ALS-inhibiting herbicides of the imidazolinone class, sulfonylurea class, and other classes. Green plants transformed with these nucleotide sequences are also resistant to derivatives of these herbicides, and to at least some of the other herbicides that normally inhibit acetohydroxyacid synthase (AHAS), particularly imidazolinone and sulfonylurea herbicides.

These nucleotide sequences may be used to transform green plants generally, particularly crop plants, both dicots and monocots. Alternatively, analogous double mutations may be introduced into green plants by site-directed mutagenesis of the plant's native ALS coding sequence(s). No marker gene is needed to select for such a transformation, since selection may be performed directly for the herbicide resistance trait itself.

The novel “double mutant” sequences, with point mutations at amino acids 197 and 574, show surprising properties that could not have been predicted from the earlier reports. The degree of herbicide resistance imparted by novel “double mutant” allele is comparable to (and in some cases may be greater than) the highest levels of resistance to ALS-inhibiting herbicides that have previously been reported. The underlying reason is currently unknown. It could not previously have been predicted that this would be the case, or that instead levels of herbicide resistance might not plateau for the “stronger” of the two point mutations, or the combination would not have been antagonistic rather than synergistic.

It is also expected that other amino acid substitutions at positions 197 and 574 will also lead to resistance to ALS-inhibiting herbicides, including mutations that could not result from single-nucleotide substitutions in the coding sequence, and would therefore be unlikely to result from undirected natural selection, or even from undirected mutation breeding efforts. However, these mutations could be induced by site-directed mutagenesis techniques known in the art. See, e.g., R. Higuchi, “Recombinant PCR,” pp. 177-183 in M. Innis et al. (Eds.), PCR Protocols: A Guide to Methods and Applications, Academic Press (1990); U.S. Pat. No. 6,010,907; Kunkel, Proc. Natl. Acad. Sci. USA, vol. 82, pp. 488-492 (1985); Kunkel et al., Methods Enzymol., vol. 154, pp. 367-382 (1987); U.S. Pat. No. 4,873,192; Walker et al. (Eds.), Techniques in Molecular Biology (MacMillan, New York, 1983); or the Genoplasty™ protocols of ValiGen (Newtown, Pa.).

The mutations described here may be incorporated into the genome of a green plant using site-directed mutagenesis, or using a transformation vector, such as those known in the art. If a transformation vector is used, the encoded ALS molecule may otherwise be native to the same plant species that is being transformed, or it may be derived from another plant, for example the Coreopsis sequences reported here. One embodiment of this aspect of the invention is a green plant comprising an oligonucleotide sequence encoding an ALS molecule identical to a wild-type ALS molecule from the same species, except for site-directed mutagenesis of the codons for amino acids 197 and 574. If the double-mutant ALS molecule is otherwise native to the plant in which it is expressed, except for the point mutations at positions 197 and 574, then such a plant should not be considered a “genetically modified organism,” in the popular sense of an organism that has been artificially transformed with an oligonucleotide coding sequence derived from a different species. For example, such a plant could be a maize plant containing an oligonucleotide sequence encoding an AHAS molecule identical to the wild-type maize ALS molecule, except for site-directed point mutations at both codons 197 and 574.

### BRIEF DESCRIPTION OF THE DRAWINGS

FIGS. 1(a) through (f) depict ALS or AHAS enzyme activity in mutant and wild type strains of C. tinctoria, and in mutant and wild type strains of rice, in the presence of varying concentrations of Arsenal, Glean, and Oust herbicides as inhibitors.

### MODES FOR CARRYING OUT THE INVENTION

DNA and amino acid sequences of the ALS gene of cocklebur (Xanthium sp.) were aligned with those from 14 other plant species. Conserved homologous regions were identified, and primers for PCR amplification were designed based on those conserved regions. The primers amplified 1.11 kb portions from the ALS sequences of both resistant mutant and susceptible wild-type C. tinctoria. The PCR-amplified ALS fragments from both mutant and wild-type plants were cloned, sequenced and compared with known ALS sequences from other plants by NCBI BLAST procedures (http://www.ncbi.nih.gov/BLAST/). The results were consistent with an ALS gene fragment. Flanking upstream sequences were then obtained. The 3′ RACE method was used to amplify the cDNA 3′ ends of the ALS genes using a 5′ sequence beginning with the start codon as the primer. The entire coding region of the ALS gene was then sequenced from both resistant and susceptible C. tinctoria. Complete ALS coding sequences were obtained from four susceptible wild-type (ALS1S1-4) and four resistant mutant strains (Race, or Genotype, 2, 3, 6, 9). All coding sequences were 1971 bp, except that sequence ALS1S2 was 1974 bp. (Nomenclatural note: As used in the specification and claims, the term “Races” refers to variations in PCR products that were identified through the “3′ RACE” PCR method. The term does not necessarily imply the presence of a “race” or “variety” in the sense of an isolated breeding population.)

A total of 164 single nucleotide polymorphisms (SNPs) were identified among the resistant and susceptible strains. Among the 164 SNPs, 124 were neutral nucleotide polymorphisms resulting in no change in amino acid sequence, while 40 resulted in amino acid substitutions. The 40 latter SNPs produced 32 different amino acid polymorphisms. Mutations identified in four of the resistant clones included Pro197 to Leu, and Trp574 to Leu. One of the resistant clones (i.e., one of the amplified PCR products) contained a novel combination of mutations, at both Pro197 and Trp574. This combination has not previously been reported. This clone exhibited strong resistance to at least the imidazolinone imazapyr [Arsenal®] and the sulfonylureas chlorsulfuron [Glean®] and sulfometuron methyl [Oust®]. The breadth and strength of herbicide resistance is atypical compared with ALS mutants that have been reported for other plants. Both the susceptible and resistant C. tinctoria ALS also showed a Ser653 to Ala shift (as compared to reported wild-type ALS for most other plants previously reported).

### Example 1

Plant materials: Herbicide susceptible (wild type) and resistant (mutant) strains of C. tinctoria, and susceptible Cocodrie rice (CCDR) and resistant CL161 rice varieties were used in this study. Seeds of Cocodrie rice, CL161 rice, and wild type C. tinctoria are available commercially.

To confirm herbicide resistance, and to characterize herbicide resistance profiles, seeds from the roadside-harvested, putatively-resistant Coreopsis were harvested (F₀) and planted in a field in a separate location from the original roadside stand. The resulting F₀ plants were not subjected to herbicide treatment. F₁ seeds were harvested from the F₀ plants, and the F₁ seeds were planted in a field in several blocks and replicates. The resulting F₁ plants were treated with various AHAS-inhibiting herbicides.

Commercial Coreopsis tinctoria seed were obtained as controls from two commercial nurseries. The commercially-obtained seed and the experimental seed were planted with a small grain drill at a seeding rate of 2 pounds per acre. Three months later, herbicides were applied to the F₁ plants at different rates in a randomized complete block design with three replications. The ALS-inhibiting herbicides used in this trial were sulfometuron methyl (trade name Oust™); imazapic (trade name Plateau™); sulfosulfuron, 1-(2-ethylsulfonylimidazo[1,2a]pyridin-3-ylsulfonyl)-3-(4,6-dimethoxypyrimidin-2-yl) urea (trade name Outrider™); and DPX 6447.

Percent injury ratings were taken at 23 and 49 days after herbicide application. All herbicide treatments injured the susceptible Coreopsis. The most severe injury to control plants was noted from the Oust applied at 0.07 pounds active ingredient per acre (ai/A), with an injury rating of 98%. The lowest injury rating for controls was from the DPX 6447 applied at 0.3 pounds ai/A, with an injury rating of 13%.

Seed were harvested from mature Coreopsis with a combine from plantings both inside and outside the herbicide treated areas at the Idlewild Research Station in Clinton, La. Seed were also harvested with a combine from the roadside area near Chase, La. Putative herbicide-resistant seed from the Chase location, and putative herbicide-sensitive seed from the Idlewild Research Station were separately cleaned and stored in a climate-controlled seed storage facility.

Seed samples from both seed sources were planted in March at the Idlewild Research Station using a small grain drill, at a 2 pound per acre seeding rate. Area one contained both control seed grown the previous season at the Idlewild Research Station, and seed obtained from the Chase, La. site. A second area was planted in March exclusively with seed from Chase, La. A third area was planted in April with seed from Chase, La.

A single application of Oust at 0.07 pounds ai/A was applied in mid-May to half of the first and third plantings and to all of the second planting. The Idlewild-grown control seed had an injury rate of 98%, while the seed from Chase had an injury rate less than 1%. A second application of Oust at 0.14 pounds ai/A was applied to part of the second planting, with no noticeable effect.

A transplanting trial was also conducted in May at the Idlewild Research Station. Five-inch diameter divots of plants were collected at both the Chase and Idlewild sites. Divots were transplanted to a location at Idlewild which had had no previous Coreopsis crop. Divots were transplanted in a paired arrangement on 36 inch centers. Oust, Plateau and Outrider were applied at various rates 2 weeks after transplant. The trial used a randomized complete block design with 3 replications. Visual injury and height ratings were taken 2 and 4 weeks after application. Injury to the susceptible transplants from Idlewild ranged from a high of 100% at all rates of Oust to a low of 60% with the 0.06 pound ai/A rate of Outrider. No injury was observed, nor any height differences noted in any application made to the resistant transplants from the Chase site.

Samples of herbicide-resistant Coreopsis tinctoria seed grown at Idlewild, derived from the Chase site, were deposited with the American Type Culture Collection (ATCC), 10801 University Boulevard, Manassas, Va. 20110-2209 on Jan. 15, 2002; and were assigned ATCC Accession No. PTA-3981.

Additionally, samples of CL161 rice seed, also known as PWC16, were deposited with ATCC on Nov. 2, 1999, and were assigned ATCC Accession No. PTA-904.

Each of the ATCC deposits described above was made pursuant to a contract between ATCC and the assignee of this patent application, Board of Supervisors of Louisiana State University and Agricultural and Mechanical College. Each of the contracts with ATCC provides for permanent and unrestricted availability of these seeds or the progeny of these seeds to the public on the issuance of the U.S. patent describing and identifying the deposit or the publication or the laying open to the public of any U.S. or foreign patent application, whichever comes first, and for the availability of these seeds to one determined by the U.S. Commissioner of Patents and Trademarks (or by any counterpart to the Commissioner in any patent office in any other country) to be entitled thereto under pertinent statutes and regulations. The assignee of the present application has agreed that if any of the seeds on deposit should become nonviable or be lost or destroyed when cultivated under suitable conditions, they will be promptly replaced on notification with a viable sample of the same seeds.

Each of the Coreopsis genotypes described in this specification derived from the original collection of seeds harvested near Chase, La. At the time the ATCC deposit was originally made, the different genotypes described here had not been identified. A large sampling of flowers was collected for the ATCC deposit. It is therefore considered to be statistically highly likely that each of the herbicide-resistant genotypes (Races 2, 3, 6, 9) is represented in the ATCC deposit described above, although this assumption has not been experimentally confirmed as of the priority date of this application. These seeds were from highly resistant plants, as their parents had been sprayed with both Oust and Arsenal to remove off-types. Herbicide resistance cuts across the various resistant genotypes described here, although levels of cross-resistance may vary from one to another. The genotypes differ from one another phenotypically in shape, flower color, and days to maturity.

### Example 2

Response of resistant and susceptible C. tinctoria to imidazolinone and sulfonylurea herbicides treatments: Both wild-type and mutant strains of C. tinctoria were planted in the greenhouse with ˜100 seeds per pot. Individual plants were transplanted to separate pots after seven weeks. The greenhouse regime comprised natural light with temperatures of 25 to 30° C. during the day and 15 to 20° C. at night. The experimental setup was a completely randomized design with five treatments and six plants (replications) per treatment for both wild-type and mutant strains. The five treatments were Arsenal (imazapyr), Oust (sulfometuron methyl), Arsenal+Oust sequential, Oust+Arsenal sequential, and control (no herbicide). Arsenal and Oust were applied with a back pack sprayer to individual mutant or susceptible, 14-week plants at rates of 8 oz. active ingredient/Acre rate and 2 oz. a.i./A, respectively, with 12 plants per each herbicide treatment for both wild-type and mutants. Ten days later, the same rate of Oust was sprayed on six plants that had previously been treated with Arsenal, and the same rate of Arsenal was sprayed on six plants that had previously been treated with Oust, for both wild-type and mutants. Plant heights and herbicide symptom ratings (0=no damage to 9=dead plant) were recorded immediately prior to the initial spraying, immediately prior to the second spraying, and 11 days after the second spraying.

### Example 3

ALS enzyme Assay: An ALS enzyme assay was used to determine if resistance in the mutants was due to the ALS enzyme itself in the mutant plants. A modification was used of the colorimetric method of Singh B. K., Stidham M. A., and Shaner D. L. 1988. Assay of acetohydroxyacid synthase. Analytical Biochemistry 171:173-179. Rice plants were used as controls. Sulfometuron methyl (Oust), chlorsulfuron (Glean), and imazapyr (Arsenal) were used as ALS inhibitors. Briefly, 10 grams of young leaves pooled from either mutant plants or from susceptible plants were ground to a powder in liquid nitrogen, and mixed with 50 ml of extraction buffer containing 100 mM K₂HPO₄ (pH 7.5), 10 mM sodium pyruvate, 5 mM EDTA, 1 mM valine, 1 mM leucine, 10 mM cysteine, 0.1 mM flavin adenine dinucleotide, 5 mM MgCl₂, 10% (v/v) glycerol, and 1% (w/v) polyvinylpyrrolidone. The homogenate was filtered through eight layers of cheesecloth and centrifuged at 25,000 g at 4° C. for 20 min. The supernatant was brought to 50% saturation with (NH₄)₂SO₄ by adding an equal volume of 100% saturated extraction buffer, and was then allowed to stand for 20-30 min on ice. The resulting solution was centrifuged at 25,000 g at 4° C. for another 20 min. The pellet was dissolved in 10 ml resuspension buffer containing 100 mM K₂HPO₄ (pH7.5), 1 mM EDTA, 100 mM NaCl, and 10% (v/v) glycerol. Bradford Reagent (SIGMA, Saint Louis, Mo., USA) was used to determine the concentration of total protein in solution; and resuspension buffer was used to adjust the concentrations of total protein from wild- and mutant-type of C. tinctoria to the same level.

ALS activity was measured by mixing 1.5 ml of assay buffer, containing 100 mM K₂HPO₄ (pH 7.0), 10 mM MgCl₂, 100 mM sodium pyruvate, 1 mM thiamine pyrophosphate, 0.1 mM flavin adenine dinucleotide, and 0.5 mL of the ALS extract, with varying amounts of herbicide (0.0.1, 1, 10, 100, 1000 μM). The mixture was then incubated at 37° C. for 1 h. The reaction was acidified by adding 6 NH₂SO₄ to a final concentration of 0.5%, and the reaction mixture was then incubated at 60° C. for 30 min to facilitate decarboxylation of acetolactate to acetoin. In a blank sample for comparison, the same concentration of KOH was added. After acidification, 1 mL was added of a freshly-prepared solution of 1-naphthol and creatin (Sigma) dissolved in 2.5 N NaOH (0.09 and 0.009 g/l respectively). The mixtures were incubated at 37° C. for 30 to 40 min for color development. The mixtures were then centrifuged at 12,000 g for 10 min, and absorbance was measured at 530 nm with a Spectrophotometer 690.

### Examples 4 and 5

Extraction of DNA and the design of PCR primers for amplifying ALS gene fragments: One or two leaves from each of about 200 individual wild-type plants were collected and combined to form a “susceptible” pooled sample. One or two leaves from each of about 200 individual mutant plants were collected and combined to form a “resistant” pooled sample. The two pooled samples were ground separately with liquid nitrogen, and total genomic DNA was isolated from each using an UltraClean™ Plant DNA Isolation Kit (MoBio Laboratories, Inc., Solana Beach, Calif., USA). DNA and amino acid sequences of the ALS gene of cocklebur (Xanthium sp.) were obtained from GenBank (http://www.ncbi.nlm.nih.gov/entrez/viewer.fcgi?db=nucleotide&val=563246; Bernasconi et al. 1995) and aligned with those of 14 other plant species: Bidens pilosa L. (Genbank Accession AJ514936), Gossypium hirsutum L. (Z46960, Z46959), Arabidopsis thaliana L. Heynh. (AY124092, X51514, AL133315, AY042819, NM114714), Bassia scoparia L. (AF094326), Solanum ptychanthum (AF308650, AF308649, AF308648), Amaranthus sp. (U55852), Amaranthus powellii (AF363370, AY094592), Amaranthus retroflexus (AF363369), Nicotiana tabacum L., tobacco, (X07644, X07645), Camelina microcarpa (AY428879, AY428880, AY4288947), Lotus corniculatus var. japonicus (AP006351), Medicago truncatula (AC141866), Zea mays (X63554), and Brassica napus (Z11524, M60068, Z11526) using the program ClustalX (ftp://ftp-igbmc.u-strasbg.fr/pub/ClustalX/). Conserved regions of the ALS gene were identified, and primers (designated AHAS3F and AHAS3R) were designed based on those conserved regions using the program Oligos© v.6.2 (http://www.biocenter.helsinki.fi/bi/bare-1html/oligos.htm, now called FastPCR©) (Table 1).

TABLE 1
PCR primers

| Primers | Sequence 5′→3′ | |
|---|---|---|
| AHAS3F | TTG GAA CAG ATG CGT TTC | SEQ ID NO. 1 |
| | AAG | |
| AHAS3R | CTC CAA CTG AAC CAC CAT | SEQ ID NO. 2 |
| | ACC CA | |
| TSP1 | TCG CAT AAA CCG TCC CGT | SEQ ID NO. 3 |
| | G | |
| TSP2 | ATC ACT CGA AGC CGG GAA | SEQ ID NO. 4 |
| | CGC T | |
| TSP3 | TCA TCC CAT TTC GGG ACC | SEQ ID NO. 5 |
| | A | |
| TSP10 | AGA TAA CCC GGT AAC CTC | SEQ ID NO. 6 |
| | ATC G | |
| TSP11 | GAT CAT TCT CCG CGG GAC | SEQ ID NO. 7 |
| | TTG ACC | |
| TSP12 | GGC CGG AAG TGG CTA TGC | SEQ ID NO. 8 |
| | AC | |
| P3F | ATG GCG GCT GTC ACT CCC | SEQ ID NO. 9 |
| P5F | CCA ACC CTA GAC ACC ATT | SEQ ID NO. 10 |
| | CAA AC | |
| ALS1R3 | CCC CGT ACA TAA CAC TCT | SEQ ID NO. 11 |
| | ATA TGC G | |

### Example 6

Isolation and sequencing of ALS gene fragments: Using the designed primers, ALS gene fragments were amplified from both mutant and wild-type strains of C. tinctoria. FailSafe™ PCR PreMix Selection Kit (EPICENTRE, Madison, Wis., USA) was used to identify a pre-mix for optimal amplification. Each 50 μL PCR reaction contained 75 ng of template DNA, 0.2 μM of each primer, 0.5 μL FailSafe™ PCR Enzyme Mix, and 25 μL of each of 12 FailSafe™ PCR 2× Premix solutions. The PCR was run on an iCycler™ Thermal Cycler (Bio-Rad, Hercules, Calif., USA) with the following thermocycle profile: 1 cycle of 94° C. for 2 min; 35 cycles of 94° C. for 45 sec, 53.5° C. for 45 sec, and 72° C. for 1 min 8 sec; and 1 cycle of 4° C. for holding. Amplified DNA products were separated on 1% agarose gels at 80 V for 1 h in 1×TAE buffer (40 mM Tris base, pH 8.0, 20 mM glacial acetic acid, 2 mM Na₂EDTA), stained with ethidium bromide, and visualized under UV light. Fragments of the expected size (AHAS3, 1.11 kb) were recovered from the agarose gel using Zymoclean™ Gel DNA Recovery Kit (Zymo Research Corporation, Orange, Calif., USA). The fragments were cloned into a TOPO TA Cloning® kit (Invitrogen Corp., Carlsbad, Calif., USA). The inserts were sequenced and compared with cocklebur and other known ALS sequences by NCBI BLAST (http://www.ncbi.nlm.nih.gov/BLAST/) procedures.

### Examples 7-9

Isolation of additional regions of the ALS gene. DNA Walking SpeedUp™ Premix Kit (Seegene USA, Rockville, Md., USA) was used to obtain adjacent upstream and downstream sequences. Based on the 1.11 kb AHAS3 fragment, three target-specific primers (TSP1, TSP2 and TSP3) were designed using the program FastPCR© v.3.6 (http://www.biocenter.helsinki.fi/bi/bare-1_html/oligos.htm) for upstream cloning (Table 1). For the first PCR reaction, each 50 μL PCR reaction contained 75 ng of template DNA derived from mutant-type of C. tinctoria, 0.2 μM of one of the four DW-ACP, 0.2 μM of primer TSP1, and 25 μL of 2×SeeAmp™ ACP™ Master Mix II. The PCR tube was placed in a preheated (94° C.) iCycler™ Thermal Cycler (Bio-Rad, Hercules, Calif., USA) with the following thermocycle profile: 1 cycle of 94° C. for 5 min; 1 cycle of 40° C. for 1 min; 1 cycle of 72° C. for 2 min; 30 cycles of 94° C. for 30 sec, 55° C. for 30 sec, and 72° C. for 1 min 40 sec, 1 cycle of 72° C. for 7 min, and 1 cycle of 4° C. for holding. The PCR products were purified using QIAquick® PCR Purification Kit (QIAGEN Inc., Valencia, Calif., USA) to remove the DW-ACP and TSP1 primers present in the first PCR reaction. For the second PCR reaction, each 20 μL PCR reaction contained 5 μL purified first PCR products, 0.5 μM DW-ACPN, 0.5 μM of primer TSP2, and 10 μL of 2×SeeAmp™ ACP™ Master Mix II. The PCR tube was placed in a preheated (94° C.) iCycler™ Thermal Cycler with the following thermocycle profile: 1 cycle of 94° C. for 3 min; 35 cycles of 94° C. for 30 sec, 60° C. for 30 sec, and 72° C. for 1 min 40 sec; 1 cycle of 72° C. for 7 min; and 1 cycle of 4° C. for holding. For the third PCR reaction, each 20 μL PCR reaction contained 2 μL of second PCR products, 0.5 μM of Universal primer, 0.5 μM of primer TSP3, and 10 μL of 2×SeeAmp™ ACP™ Master Mix II. The PCR tube was placed in a preheated (94° C.) iCycler™ Thermal Cycler with the same thermocycle profile as in the second PCR reaction. Amplified DNA products were separated on 1% agarose gels at 80 V for 1 h in 1×TAE buffer (40 mM Tris base, pH 8.0, 20 mM glacial acetic acid, 2 mM Na₂EDTA), stained with ethidium bromide, and visualized under UV light. The fragments were recovered from agarose gel using Zymoclean™ Gel DNA Recovery Kit (Zymo Research Corporation, Orange, Calif., USA) and cloned into a TOPO TA Cloning® kit (Invitrogen Corp., Carlsbad, Calif., USA). The inserts were sequenced and compared with cocklebur and other known ALS sequences by NCBI BLAST (http://www.ncbi.nlm.nih.gov/BLAST/) procedures.

The result was a 202 bp upstream fragment that did not span the entire upstream coding region. Three additional target-specific primers (TSP10, TSP11, and TSP12) were then designed for further upstream cloning (Table 1). The procedures used were same as for the first set of three target-specific primers (TSP1, TSP2 and TSP3), except that the thermocycle profile for the first PCR was 1 cycle of 35° C. for 1 min instead of cycle of 40° C. for 1 min. A 1.82 kb upstream fragment was obtained, which covered the entire upstream and promoter (1.381 kb) regions.

### Example 10

Extraction of RNA, and isolation of the entire coding region from mutant ALS genes. Pooled samples of young leaves were prepared as in Examples 4 and 5. Total RNA was isolated from the pooled samples using an RNeasy® Plant Mini Kit (QIAGEN Inc., Valencia, Calif., USA). An RNase-Free DNase Set (QIAGEN Inc., Valencia, Calif., USA) was used for on-column digestion of DNA during RNA purification. A commercial kit, “3′ RACE System for Rapid Amplification of cDNA Ends” (Invitrogen Corp., Carlsbad, Calif.), was used to amplify the cDNA 3′ ends of the ALS genes using a 5′ sequence beginning with the start codon as the primer (P3F) (Table 1). Total RNA (2.45 μg) was used for first strand cDNA synthesis. In amplifying target cDNA, a high-fidelity thermostable PCR enzyme, Platinum® Pfx DNA Polymerase (Invitrogen Corp., Carlsbad, Calif.), was used. Each 25 μL PCR reaction contained 1 μL of the cDNA synthesis reaction solution as template, 1×pfx Amplification Buffer, 1 mM MgSO₄, 0.288 μM of each primer (P3F and AUAP), 0.32 mM dNTPs, and 1.25 U Platinum® Pfx DNA Polymerase. The PCR was run on an iCycler™ Thermal Cycler (Bio-Rad, Hercules, Calif., USA) with the following thermocycle profile: 1 cycle of 94° C. for 3 min; 35 min; and 1 cycle of 4° C. for holding. Amplified DNA products were separated on 1% agarose gels at 80 V for 1 h, in 1×TAE buffer (40 mM Tris base, pH 8.0, 20 mM glacial acetic acid, 2 mM Na₂EDTA), stained with ethidium bromide and visualized under UV light. Fragments of the expected size (˜2 kb) were recovered from agarose gel using Zymoclean™ Gel DNA Recovery Kit (Zymo Research Corporation, Orange, Calif., USA). The fragments were cloned into the TOPO TA Cloning® kit (Invitrogen Corp., Carlsbad, Calif., USA). Before ligation, 3′ A-overhangs were added to the purified PCR fragments. Briefly, a 15 μL reaction contained 11.6 μL of purified PCR products (10 ng/μL), 1×PCR Reaction Buffer, 2.5 mM MgCl₂, 0.2 mM dNTPs, and 0.5 U Taq DNA polymerase (Promega Corp., Madison, Wis., USA). The reaction was carried out at 72° C. water bath for 12 min. The inserts were sequenced and compared with cocklebur and other known ALS sequences by NCBI BLAST (http://www.ncbi.nlm.nih.gov/BLAST/) procedures.

### Example 11

Isolation of entire coding region of wild-type ALS Genes. PCR was carried out using wild-type C. tinctoria DNA as the template. The forward primer, P5F, was immediately upstream of the ATG start codon, and the reverse primer, ALS1R3 was immediately downstream of the TGA stop codon (Table 1). The sequence 5′-CCC CG was inserted at the 5′ end of ALS1R3 as an adaptor to increase the annealing temperature to 57° C. (Table 1). A high fidelity thermostable PCR enzyme, Platinum® Pfx DNA Polymerase (Invitrogen Corp., Carlsbad, Calif.), was used in the PCR reactions. Each 15 μL PCR reaction contained 23 ng of wild-type C. tinctoria DNA, 1×pfx Amplification Buffer, 1 mM MgSO₄, 0.293 μM of each primer (P5F and ALS1R3), 0.33 mM dNTPs, and 0.75 U Platinum® Pfx DNA Polymerase. The PCR was run on an iCycler™ Thermal Cycler (Bio-Rad, Hercules, Calif., USA) with the following thermocycle profile: 1 cycle of 94° C. for 2 min; 1 cycle of 50° C. for 30 sec; 1 cycle of 68° C. for 2 min; 34 cycles of 94° C. for 15 sec, 60° C. for 30 sec, and 68° C. for 2 min; 1 cycle of 68° C. for 5 min; and 1 cycle of 4° C. for holding. Amplified DNA products were separated on 1% agarose gels at 80 V for 1 h in 1×TAE buffer (40 mM Tris base, pH 8.0, 20 mM glacial acetic acid, 2 mM Na₂EDTA), stained with ethidium bromide and visualized under UV light. Fragments of the expected size (˜2 kb) were recovered from agarose gel using Zymoclean™ Gel DNA Recovery Kit (Zymo Research Corporation, Orange, Calif., USA). The fragments were cloned into a TOPO TA Cloning® kit (Invitrogen Corp., Carlsbad, Calif., USA). The cloning procedures were the same as those for the 3′ Race. The inserts were sequenced and compared with cocklebur and other known ALS sequences by NCBI BLAST (http://www.ncbi.nlm.nih.gov/BLAST/) procedures.

### Examples 12-17

### Response of Resistant and Susceptible C. tinctoria Plants to Herbicide Treatments

Table 2 shows that susceptible C. tinctoria plants sprayed with sulfosulfuron methyl, or imazapyr, or a combination of both sustained a 68-89% reduction in plant height 25 days after treatment as compared to unsprayed, susceptible controls. The combined imazapyr+sulfosulfuron methyl treatment impaired growth less than either herbicide used alone. The range of herbicide injury ratings for the sprayed susceptible plants was 3.3 to 4.8, with essentially no injury for the unsprayed controls. By forty-five days after herbicide treatment, all susceptible plants had died; while all resistant plants were green, healthy, and showed little sign of herbicide damage.

TABLE 2
Mean plant height and injury rating for susceptible and resistant
C. tinctoria sprayed with sulfosulfuron methyl and imazapyr herbicides

| | Plant | % height reduction | |
|---|---|---|---|
| | height | compared to no | Injury |
| Herbicide treatment | (cm) | spray control | rating** |
| Susceptible plants* | | | |
| Sulfosulfuron methyl (SM)¹ | 6.2 | 89 | 3.3 |
| Imazapyr (IM)² | 11.0 | 80 | 3.5 |
| SM + IM³ | 11.7 | 79 | 4.8 |
| IM + SM⁴ | 17.6 | 68 | 3.5 |
| No spray | 55.2 | — | 0 |
| Resistant plants* | | | |
| Sulfosulfuron methyl (SM)¹ | 10.8 | 0 | 0.5 |
| Imazapyr (IM)² | 9.2 | 0 | 0.7 |
| SM + IM³ | 11.6 | 0 | 1.2 |
| IM + SM⁴ | 30.2 | 0 | 0.7 |
| No spray | 9.2 | — | 0 |

¹SM sprayed at 8 oz. a.i./A
²IM sprayed at 2 oz. a.i./A
³SM + IM: SM sprayed on March 3 and IM on March 13
⁴IM + SM: IM sprayed on March 3 and SM on March 13
*Six plants were evaluated for each treatment
**Injury rating, where 0 = no injury, and 10 = dead plant

Plant height in resistant plants was not affected when sprayed with sulfosulfuron methyl or imazapyr, as compared to the unsprayed resistant controls. The height of the resistant plants actually increased 3-fold after the imazapyr+sulfosulfuron methyl sequential sprays (Table 2). It appeared that the imazapyr+sulfosulfuron methyl treatment stimulated growth in the resistant plants, an observation that will be confirmed in follow-up experiments. Various strains or genotypes of C. tinctoria were used in this study, so a direct evaluation of herbicide on plant height should be viewed with caution. However, a clear, general trend was that the resistant plants showed far greater resistance to herbicide than the susceptible plants. Herbicide injury ratings for the resistant plants ranged from 0.5 to 1.2 (Table 2). The resistant plants and the unsprayed control plants both showed essentially no injury, while after 45 days all susceptible plants were dead.

### Examples 18-20

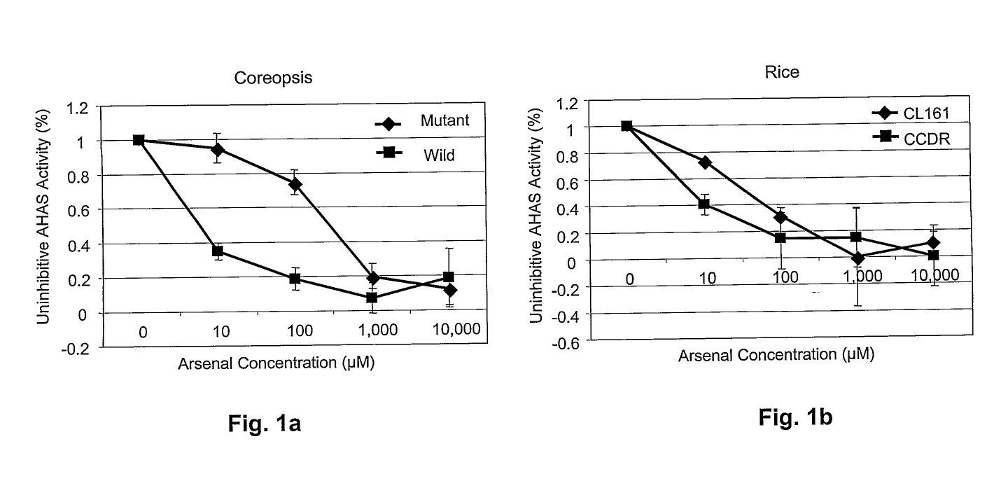

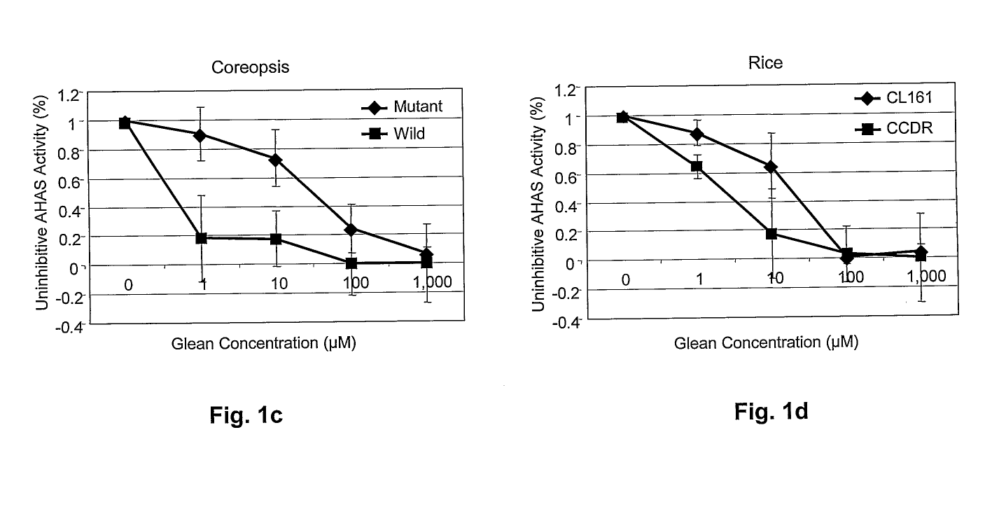

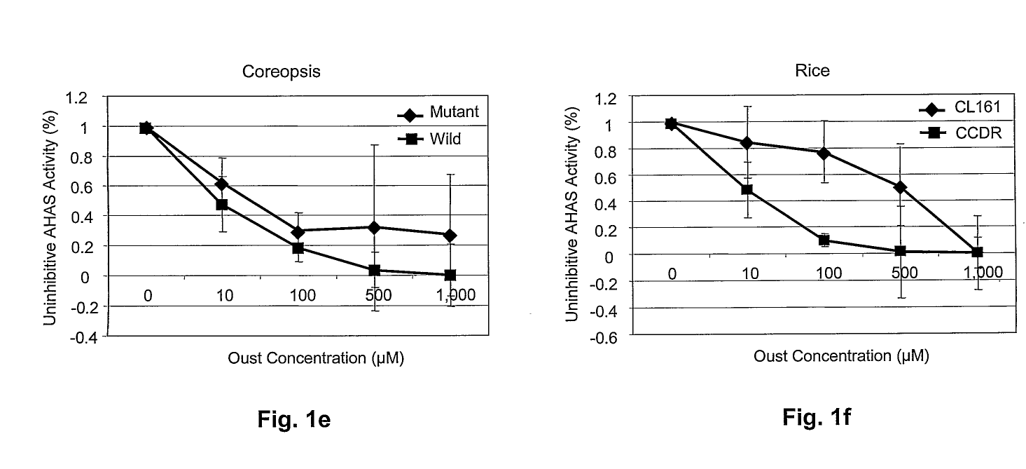

AHAS/ALS enzyme activity. FIGS. 1(a) through (f) depict ALS enzyme activity in mutant and wild type strains of C. tinctoria, and in mutant and wild type strains of rice, in the presence of several concentrations of Arsenal, Glean, and Oust herbicides as inhibitors. Results from the enzyme analyses showed that the ALS enzyme from the resistant C. tinctoria mutants were ˜2.5-fold more tolerant to Arsenal herbicide (imazapyr) at 10 to 100 μM concentrations as compared to that from the susceptible variety (FIG. 1a). By contrast, the enzyme from CL161 rice was <2-fold more tolerant at the 10 μM concentration (FIG. 1b). The C. tinctoria mutant AHAS exhibited ˜4.5-fold greater tolerance to Glean herbicide (chlorsulfuron) than the susceptible control at 1 to 10 μM concentrations (FIG. 1c). The CL161 rice variety AHAS was <1-fold more tolerant than control at 1 μM concentration (FIG. 1d). The C. tinctoria mutants were more tolerant than the susceptible control for Oust herbicide (sulfometuron methyl) (FIG. 1e), although the difference was not significant. The CL161 rice exhibited significantly greater tolerance than the susceptible variety CCDR at 100 μM of Oust herbicide (sulfometuron methyl) (FIG. 1f).

### Examples 21-22

ALS sequence analysis of 1.11 kb fragment and promoter region. The conserved region isolated with the primers AHAS3F and AHAS3R (Table 1) was 1.11 kb. A search of the DNA sequence databases in the GenBank showed that the 1.11 kb C. tinctoria sequence contained significant sequence similarity to other higher plant ALS sequences (data not shown). The 1.381 kb promoter and upstream untranslated region of the C. tinctoria ALS sequence exhibited the characteristic “TATATT” box of eukaryotic promoters beginning at nucleotide 1330.

### Examples 23-27

Nucleotide polymorphisms among C. tinctoria ALS sequences associated with herbicide resistance. Table 3 below identifies the various DNA sequences and inferred amino acid sequences, from resistant and susceptible Coreopsis tinctoria.

TABLE 3

| | |
|---|---|
| SEQ ID NO 12 | ALS Promoter Region |
| SEQ ID NO 13 | ALS DNA Coding Sequence, Race 2, |
| | Resistant |
| SEQ ID NO 14 | ALS DNA Coding Sequence, Race 3, |
| | Resistant |
| SEQ ID NO 15 | ALS DNA Coding Sequence, Race 6, |
| | Resistant |
| SEQ ID NO 16 | ALS DNA Coding Sequence, Race 9, |
| | Resistant |
| SEQ ID NO 17 | ALS Inferred Amino Acid Sequence, |
| | Race 2, Resistant |
| SEQ ID NO 18 | ALS Inferred Amino Acid Sequence, |
| | Race 3, Resistant |
| SEQ ID NO 19 | ALS Inferred Amino Acid Sequence, |
| | Race 6, Resistant |
| SEQ ID NO 20 | ALS Inferred Amino Acid Sequence, |
| | Race 9, Resistant |
| SEQ ID NO 21 | ALS DNA Coding Sequence, ALS1S1, |
| | Susceptible |
| SEQ ID NO 22 | ALS DNA Coding Sequence, ALS1S2, |
| | Susceptible |
| SEQ ID NO 23 | ALS DNA Coding Sequence, ALS1S3, |
| | Susceptible |
| SEQ ID NO 24 | ALS DNA Coding Sequence, ALS1S4, |
| | Susceptible |
| SEQ ID NO 25 | ALS Inferred Amino Acid Sequence, |
| | ALS1S1, Susceptible |
| SEQ ID NO 26 | ALS Inferred Amino Acid Sequence, |
| | ALS1S2, Susceptible |
| SEQ ID NO 27 | ALS Inferred Amino Acid Sequence, |
| | ALS1S3, Susceptible |
| SEQ ID NO 28 | ALS Inferred Amino Acid Sequence, |
| | ALS1S4, Susceptible |

A comparison of mutant and wild-type C. tinctoria ALS sequences revealed that all eight cloned sequences encoded Ala653. See Table 4. Among the mutant C. tinctoria, the Race 2 clone exhibited a nucleotide change from G1682 to T1682, resulting in an amino acid change from Trp574 to Leu574 (Table 4). Race 9 had a nucleotide change from C548 to T548, resulting in an amino acid change from Pro197 to Leu197 (Table 4). Race3 had both Pro197→Leu197 and Trp574→Leu574 changes (Table 4). Although Race 6 was obtained from the resistant pool of Coreopsis leaves, we identified no mutation sites in the ALS sequence from Race 6 appearing to confer resistance to ALS-inhibiting herbicides, as compared to the four sequences (ALS1S1-4) from wild-type C. tinctoria (Table 4). Compared to wild-type ALS gene sequences of C. tinctoria, Race 2 and 3 exhibited putative mutations for cross-tolerance to different ALS-inhibiting herbicides. Race 9 showed putative resistance mutations for sulfonylureas (Table 4).

Race 3 was unique in harboring two mutations in the same allele, one mutation associated with cross-tolerance, and one associated with sulfonylurea herbicide resistance. Enzyme assays, greenhouse tests, and field tests, otherwise along the lines generally described in this specification, will confirm that plants with the Race 3 genotype have greater herbicide resistance and greater cross-tolerance and than plants with the other genotypes, including the other resistant genotypes.

TABLE 4
Nucleotide polymorphisms (indicated in bold italics within the codon)
in the ALS gene sequences of C. tinctoria that resulted
in amino acid substitutions in the gene

| Nucleotide | | | |
|---|---|---|---|
| position fr om 5′ | | Amino | |
| end^{a} | Codons | acid^{b} | Sequences |
| 16, 17 | [symbol] C | Pro6 | Race2, 3, 6, 9, ALS1S1, 3, 4 |
| | [symbol] C | Asp6 | ALS1S2 |
| 23 | A [symbol] C | Asn8 | Race2, 3, 9, ALS1S1-3 |
| | A [symbol] C | Ser8 | Race6, ALS1S4 |
| 25 | [symbol] CT | Pro9 | Race2, 3, 9, ALS1S1-3 |
| | [symbol] CT | Ser9 | Race6, ALS1S4 |
| 31 | ∞TC | Ile11 | Race2, 3, 6, 9, ALS1S1, 3, 4 |
| | [symbol] TC | Phe11 | ALS1S2 |
| 76, 77, 78 | — | | Race2, 3, 6, 9, ALS1S1, 3, 4 |
| | A [symbol] [symbol] | Thr31 | ALS1S2 |
| 82, 83 | [symbol] C | Asn32 | Race2, ALS1S1, 3 |
| | TTC | Phe32 | Race3, 6, 9, ALS1S2, 4 |
| 104 | C [symbol] T | His39 | Race3, 6, 9, ALS1S2 |
| | C [symbol] T | Arg39 | Race2, ALS1S1, 3, 4 |
| 131 | C [symbol] C | Pro48 | ALS1S4 |
| | CTC | Leu48 | Race2, 3, 6, 9, ALS1S1-3 |
| 140 | C [symbol] A | Gln51 | ALS1S4 |
| | CGA | Arg51 | Race2, 3, 6, 9, ALS1S1-3 |
| 199, 200, 201 | A [symbol] | Thr79 | Race6 |
| | [symbol] | Thr79 | Race2, 3, 9, ALS1S1, 3, 4 |
| | [symbol] | Cys79 | ALS1S2 |
| 268, 270 | [symbol] AT [symbol] | Ile104 | ALS1S2 |
| | [symbol] T [symbol] | Val104 | Race2, ALS1S3, 4 |
| | [symbol] T [symbol] | Val104 | Race3, 6, 9 |
| | [symbol] T [symbol] | Val104 | ALS1S1 |
| 273 | GA [symbol] | Glu105 | Race2, 3, 6, 9, ALS1S1, 3, 4 |
| | GA [symbol] | Asp105 | ALS1S2 |
| 274 | [symbol] CC | Pro106 | ALS1S2 |
| | [symbol] CC | Ala106 | Race2, 3, 6, 9, ALS1S1, 3, 4 |
| 280 | [symbol] AA | Lys108 | ALS1S2 |
| | [symbol] AA | Glu108 | Race2, 3, 6, 9, ALS1S1, 3, 4 |
| 283 | [symbol] GC | Arg109 | Race2, 3, 6, 9, ALS1S1, 3, 4 |
| | [symbol] GC | Gly109 | ALS1S2 |
| 286 | [symbol] AA | Gln110 | ALS1S2 |
| | [symbol] AA | Glu110 | Race2, 3, 6, 9, ALS1S1, 3, 4 |
| 295 | [symbol] CC | Thr113 | Race2, 3, 6, 9, ALS1S1, 3, 4 |
| | [symbol] CC | Pro113 | ALS1S2 |
| 421 | [symbol] CC | Pro155 | ALS1S2 |
| | [symbol] CC | Ala155 | Race2, 3, 6, 9, ALS1S1, 3, 4 |
| 454 | [symbol] CC | Pro166 | ALS1S2 |
| | [symbol] CC | Ala166 | Race2, 3, 6, 9, ALS1S1, 3, 4 |
| 472 | [symbol] CT | Thr172 | ALS1S2 |
| | [symbol] CT | Ala172 | Race2, 3, 6, 9, ALS1S1, 3, 4 |
| 494 | C [symbol] T | Arg179 | ALS1S2 |
| | C [symbol] T | Leu179 | Race2, 3, 6, 9, ALS1S1, 3, 4 |
| 513 | GA [symbol] | Asp185 | ALS1S4 |
| | GA [symbol] | Glu185 | ALS1S2 |
| | GA [symbol] | Asp185 | Race2, 3, 6, 9, ALS1S1, 3 |
| 521 | C [symbol] C | Pro188 | Race2, 3, 6, 9, ALS1S1, 3, 4 |
| | C [symbol] C | Arg188 | ALS1S2 |
| 548 | C [symbol] G | Pro197 | Race2, 6, ALS1S1-4 |
| | C [symbol] G | Leu197 | Race3, 9 |
| 551, 552 | C [symbol] | Pro198 | ALS1S2 |
| | C [symbol] | Arg198 | Race2, 3, 6, 9, ALS1S1, 3, 4 |
| 831 | GA [symbol] | Glu290 | Race6, 9, ALS1S3, 4, |
| | GA [symbol] | Asp290 | Race2, 3, ALS1S1, 2 |
| 906 | GA [symbol] | Asp315 | Race6, 9, ALS1S1, 2, 4 |
| | GA [symbol] | Glu315 | Race2, 3, ALS1S3 |
| 927 | GA [symbol] | Glu322 | Race2, 3, 6, ALS1S1-4 |
| | GA [symbol] | Asp322 | Race9 |
| 974 | T [symbol] C | Tyr338 | Race6, 9, ALS1S3 |
| | T [symbol] C | Phe338 | Race2, 3, ALS1S1, 2, 4 |
| 1250 | G [symbol] T | Asp430 | ALS1S2 |
| | G [symbol] T | Gly430 | Race2, 3, 6, 9, ALS1S1, 3, 4 |
| 1387 | [symbol] GC | Gly476 | Race2, 3, 6, ALS1S1-4 |
| | [symbol] GC | Cys476 | Race9 |
| 1682 | T [symbol] G | Trp574 | Race6, 9, ALS1S1-4 |
| | T [symbol] G | Leu574 | Race2, 3 |

^{a}In this table, the nucleotide sequences of the ALS gene are numbered in reference to C. tinctoria ALS1S2, because its insertion mutation resulted in three more nucleotides than other C. tinctoria sequences.
^{b}In this table, the amino acids were numbered in correspondence to the reported sequence for A. thaliana (Sathasivan et al. 1990).

TABLE 5
Amino acid polymorphisms in the ALS gene sequences of
C. tinctoria that correspond to analogous, previously-reported,
individual point mutations associated with resistance to ALS-inhibiting
herbicides. (The individual point mutations below have been previously
reported in other plants. However, it is believed that the combination
of mutations in Race 3 has not previously been reported.)

| Sequence | Amino acid^{a} | Herbicide resistance |
|---|---|---|
| Race2 | Trp574→Leu574 | Cross-tolerant |
| | Ala653 | Imidazolinones |
| Race3 | Trp574→Leu574 | Cross-tolerant |
| | Pro197→Leu197 | Sulfonylureas |
| | Ala653 | Imidazolinones |
| Race9 | Pro197→Leu197 | Sulfonylureas |
| | Ala653 | Imidazolinones |
| Race6 | Ala653 | Imidazolinones |
| ALS1S1-4 | Ala653 | Imidazolinones |

^{a}The amino acids were numbered according to A. thaliana (Sathasivan et al. 1990)

Thus we identified two nucleotide substitutions in mutant C. tinctoria ALS genes that resulted in amino acid changes (Pro197→Leu197 and Trp574→Leu574) that confer resistance to ALS-inhibiting herbicides. Specifically, a Pro197 to Leu197 mutation was detected in Race 9, a Trp574 to Leu574 mutation was observed in Race 2, and both Pro197 to Leu197 and Trp574 to Leu574 mutations were found in Race 3. This combination in a single allele, as in Race 3, has not been reported previously. The results from the AHAS enzyme assay were consistent with the ALS DNA sequences, indicating that the AHAS locus was associated with resistance to imidazolinone and sulfonylurea herbicides.

The mutation of Pro197 to Leu197 was identified in Race 3 and 9 mutant C. tinctoria ALS genes. Amino acid substitutions at Pro197 have been identified in ALS genes of at least 13 different species (see Tranel 2006).

A Trp574 to Leu574 mutation was identified in Races 2 and 3 of the mutant C. tinctoria. A Trp574 to Leu 574 substitution has been reported in several other plant species with varieties resistant to AHAS-inhibiting herbicides. See, e.g., Tranel et al. 2006.

Two other mutations were identified with potential implications for herbicide tolerance. In Race 9 (only), we observed the previously-unreported mutations Glu322 to Asp 322 and Gly476 to Cys476. Using enzyme, greenhouse, and field assays as otherwise described in this specification, the effect of these two mutations on imidazolinone resistance, sulfonylurea resistance, and resistance to other ALS-inhibiting herbicides will be confirmed. If either or both of these mutations are confirmed to impart herbicide resistance, then the use of either or both mutations as otherwise described in this specification is considered to be within the scope of this invention—both the use of such mutation(s) alone, or in conjunction with other herbicide resistance mutations, such as Pro197 to Leu197, or Trp574 to Leu 574, or other mutations known in the art.

The eight C. tinctoria ALS sequences we analyzed varied at 164 nucleotides within the coding sequence, which accounted for a combined total of 8.3% of the entire ALS coding sequence. Among the 164 SNPs, 124 were neutral nucleotide polymorphisms, while 40 would cause amino acid substitutions, with a total of 32 different polymorphisms at the amino acid level. Except for the two mutations Pro197 to Leu197, and Trp574 to Leu574, none of the remaining 30 amino acid mutations corresponded to any changes that have previously been reported as being associated with herbicide resistance, nor are any of these 30 amino acids unique to the resistant lines.

Of particular interest for developing herbicide resistant crops through genetic engineering is Race 3, which has the herbicide resistance allele with a unique combination of two mutations within the coding sequence: Pro 197→Leu197 and Trp574→Leu574. This unique combination of mutations provides exceptionally broad-spectrum resistance to both imidazolinone and sulfonylurea herbicides.

The judicious use of Race 2 and Race 9 in conjunction with Race 3 could also be used to reduce or eliminate selection of herbicide-resistant weeds by rotation of different ALS-inhibiting herbicide classes.

The resistant ALS genes may also be used as selection markers in plant transformation techniques otherwise known in the art, e.g., as otherwise described in Kovar J. L., Zhang J., Funke R. P., and Weeks D. P. 2002. Molecular analysis of the acetolactate synthase gene of Chlamydomonas reinhardtii and development of a genetically engineered gene as a dominant selectable marker for genetic transformation. The Plant Journal 29:109-117.

It will be understood by those skilled in the art that the nucleic acid sequences of the resistant mutant Coreopsis ALS enzymes are not the only sequences that can be used to confer resistance. As previously discussed, other point mutations may be introduced at the same codons 197 and 574. In addition, any of the above may be encoded by nucleic acid sequences that encode the same amino acid sequences as otherwise described but that, because of the degeneracy of the genetic code, possess different nucleotide sequences. The genetic code may be found in numerous references concerning genetics or biology, including, for example, FIG. 9.1 on page 214 of B. Lewin, Genes VI (Oxford University Press, New York, 1997). FIGS. 9.1 and 9.3 on pages 214 and 216 of Lewin directly illustrate the degeneracy of the genetic code. For example, the (mRNA) codon for leucine may be UUA, UUG, CUU, CUC, CUA, or CUG.

The sequences may be transformed into essentially any plant of interest, for example crop plants and ornamental plants. The sequenced Coreopsis ALS genes lacked introns, a situation that simplifies their regulation in other plants.

The expression products are preferably targeted to the chloroplasts, which are believed to be the major site for wild type ALS activity in green plants. The targeting signal sequence normally corresponds to the amino terminal end of the protein expression product, and the corresponding coding sequence should therefore appear upstream of the 5′ end of the coding sequence. This targeting is preferably accomplished with the native Coreopsis ALS signal sequence, but it may also use other plant chloroplast signal sequences known in the art, such as, for example, those disclosed in Cheng et al., J. Biol. Chem., vol. 268, pp. 2363-2367 (1993); see also Comai et al., J. Biol. Chem., vol. 263, pp. 15104-15109 (1988).

The invention also encompasses nucleotide sequences encoding ALS proteins having one or more silent amino acid changes in portions of the molecule not involved with resistance or catalytic function. For example, alterations in the nucleotide sequence that result in the production of a chemically equivalent amino acid at a given site are contemplated; thus, a codon for the amino acid alanine, a hydrophobic amino acid, may be substituted by a codon encoding another hydrophobic residue, such as glycine, or may be substituted with a more hydrophobic residue such as valine, leucine, or isoleucine. Similarly, changes that result in the substitution of one negatively-charged residue for another, such as aspartic acid for glutamic acid, or one positively-charged residue for another, such as lysine for arginine, can also be expected to produce a biologically equivalent product. See, e.g., FIG. 1.8 on page 10 of Lewin (1997), showing the nature of the side chains of the “standard” 20 amino acids encoded by the genetic code. (Note also a typographical error in that published figure, namely that the abbreviation for glutamine should be “Gln.”)

The invention also encompasses chimeric nucleotide sequences, in which the mutated portion of a resistant Coreopsis ALS nucleotide sequence is recombined with unaltered portions of the ALS nucleotide sequence from another species.

This invention relates not only to a functional ALS enzyme having the amino acid sequence encoded by a mutant, resistant ALS nucleotide sequence described in this specification, including for example those from the resistant Coreopsis described above, but also to an enzyme having modifications to such a sequence resulting in an amino acid sequence having the same function (i.e., a functional ALS enzyme, with resistance to at least some herbicides that normally interfere with ALS), with point mutations at both locations 197 and 574, and otherwise having about 60-70%, preferably 90% or greater homology to the sequence of the amino acid sequence encoded by the Coreopsis ALS nucleotide sequence, most preferably about 95% or greater homology, particularly in conserved regions such as, for example, a putative herbicide binding site. “Homology” means identical amino acids or conservative substitutions (e.g., acidic for acidic, basic for basic, polar for polar, nonpolar for nonpolar, aromatic for aromatic). The degree of homology can be determined by simple alignment based on programs known in the art, such as, for example, GAP and PILEUP by GCG, or the BLAST software available through the NIH internet site. Most preferably, a certain percentage of “homology” would be that percentage of identical amino acids.

A particular desired point mutation may be introduced into an ALS coding sequence using site-directed mutagenesis methods known in the art, as described previously.

Isolated ALS DNA sequences in accordance with the present invention are useful to transform target crop plants or ornamental plants, and thereby confer resistance. A broad range of techniques exists for achieving the direct or indirect transformation of plants with exogenous DNA, and any method by which one of the novel sequences can be incorporated into the host genome, and stably inherited by its progeny, is contemplated by the present invention.

The cloned ALS coding sequence should be placed under the control of a suitable promoter, so that it is appropriately expressed in cells of the transformed plant. It is expected that the most suitable promoter would be a native ALS promoter. The native ALS promoter could be that of any plant, for example, the native ALS promoter from the plant that is being transformed. Alternatively, it is expected that the native Coreopsis AHAS promoter will function appropriately in other green plants generally (including, e.g., both monocots and dicots), and that for simplicity the same Coreopsis ALS promoter may be used when transforming any plant species of interest. The Coreopsis ALS promoter comprises bases 1 to 1360 in SEQ ID NO 12. This promoter contains the consensus “TATA” box, but otherwise appears to be unique as compared to previously reported promoters.

As a further alternative, a constitutive promoter could be used to control the expression of the transformed mutant AHAS coding sequence. Promoters that act constitutively in plants are well known in the art, and include, for example, the cauliflower mosaic virus 35S promoter.

Transformation of plant cells can be mediated by the use of vectors. A common method for transforming plants is the use of Agrobacterium tumefaciens to introduce a foreign nucleotide sequence into the target plant cell. For example, a mutant AHAS nucleotide sequence is inserted into a plasmid vector containing the flanking sequences in the Ti-plasmid T-DNA. The plasmid is then transformed into E. coli. A triparental mating is carried out among this strain, an Agrobacterium strain containing a disarmed Ti-plasmid containing the virulence functions needed to effect transfer of the ALS-containing T-DNA sequences into the target plant chromosome, and a second E. coli strain containing a plasmid having sequences necessary to mobilize transfer of the ALS construct from E. coli to Agrobacterium. A recombinant Agrobacterium strain, containing the necessary sequences for plant transformation, is used to infect leaf discs. Discs are grown on selection media and successfully transformed regenerants are identified. The recovered plants are resistant to the effects of herbicide when grown in its presence.

Plant viruses also provide a possible means for transfer of exogenous DNA.

Direct uptake of DNA by plant cells can also be used. Typically, protoplasts of the target plant are placed in culture in the presence of the DNA to be transferred, along with an agent that promotes the uptake of DNA by protoplasts. Such agents include, for example, polyethylene glycol and calcium phosphate.

Alternatively, DNA uptake can be stimulated by electroporation. In this method, an electrical pulse is used to open temporary pores in a protoplast cell membrane, and DNA in the surrounding solution is then drawn into the cell through the pores. Similarly, microinjection can be used to deliver the DNA directly into a cell, preferably directly into the nucleus of the cell.

In many of these techniques, transformation occurs in a plant cell in culture. Subsequent to the transformation event, plant cells must be regenerated to whole plants. Techniques for the regeneration of mature plants from callus or protoplast culture are known for a large number of plant species. See, e.g., Handbook of Plant Cell Culture, Vols. 1-5, 1983-1989 McMillan, N.Y.

Alternate methods are also available that do not necessarily require the use of isolated cells and plant regeneration techniques to achieve transformation. These are generally referred to as “ballistic” or “particle acceleration” methods, in which DNA-coated metal particles are propelled into plant cells by either a gunpowder charge (see Klein et al., Nature 327: 70-73, 1987) or by electrical discharge (see EPO 270 356). In this manner, plant cells in culture or plant reproductive organs or cells, e.g. pollen, can be stably transformed with the DNA sequence of interest.

Direct uptake of DNA may be used. For example, in maize or rice the cell wall of cultured cells is digested in a buffer with one or more cell wall-degrading enzymes, such as cellulase, hemicellulase, and pectinase, to isolate viable protoplasts. The protoplasts are washed several times to remove the degrading enzymes, and are then mixed with a plasmid vector containing the nucleotide sequence of interest. The cells can be transformed with either PEG (e.g. 20% PEG 4000) or by electroporation. The protoplasts are placed on a nitrocellulose filter and cultured on a medium with embedded maize cells functioning as feeder cultures. After 2-4 weeks, the cultures in the nitrocellulose filter are placed on a medium containing herbicide and maintained in the medium for 1-2 months. The nitrocellulose filters with the plant cells are transferred to fresh medium with herbicide and nurse cells every two weeks. The un-transformed cells cease growing and die after a time.

Other methods of transforming plants are described in B. Jenes et al., and in S. Ritchie et al., in S.-D. Kung et al. (Eds.), Transgenic Plants, vol. 1, Engineering and Utilization, Academic Press, Inc., Harcourt Brace Jovanovich (1993); and in L. Mannonen et al., Critical Reviews in Biotechnology, vol. 14, pp. 287-310 (1994). See also the various references cited on pages 15-17 of published international patent application WO 00/26390, each of which is incorporated by reference. Other transformation methods include vacuum infiltration and floral dip. See Clough and Bent. 1998. The Plant Journal. 16:735-743.

A particularly preferred transformation vector, which may be used to transform seeds, germ cells, whole plants, or somatic cells of monocots or dicots, is the transposon-based vector disclosed in U.S. Pat. No. 5,719,055. This vector may be delivered to plant cells through one of the techniques described above or, for example, via liposomes that fuse with the membranes of plant cell protoplasts. The use of the vector of U.S. Pat. No. 5,719,055 allows the introduction of the desired coding sequence only, without any other coding sequences being introduced into the genome. No antibiotic-resistance genes or other markers will be needed: selection for successful transformation events can be based directly on the herbicide resistance itself. As explained more fully in U.S. Pat. No. 5,719,055, the only sequences that need be introduced in addition to the nucleotide sequence of interest are flanking insertion sequences recognized by the transposase used by the vector. The insertion sequences are not themselves coding sequences, and are inert in the absence of the transposase; furthermore, the vector is designed so that the transposase is not encoded by any DNA that is inserted into the transformed chromosome. The only portion of the transformed DNA that will be active following transformation is the resistant ALS nucleotide sequence itself.

The present invention can be applied to transform virtually any type of green plant, both monocot and dicot. Among the crop plants, ornamental plants, and other plants for which transformation for herbicide resistance is contemplated are (for example) rice, maize, wheat, millet, rye, oat, barley, sorghum, sunflower, sweet potato, cassava, alfalfa, sugar cane, sugar beet, canola and other Brassica species, sunflower, tomato, pepper, soybean, tobacco, melon, lettuce, celery, eggplant, carrot, squash, melon, cucumber and other cucurbits, beans, cabbage and other cruciferous vegetables, potato, tomato, peanut, pea, other vegetables, cotton, clover, cacao, grape, citrus, strawberries and other berries, fruit trees, and nut trees. The novel sequences may also be used to transform turfgrass, ornamental species, such as petunia and rose, and woody species, such as pine and poplar.

Miscellaneous

It is expected that the resistant Coreopsis lines will show resistance to ALS-inhibiting herbicides other than those tested to date. In addition to those discussed above, such herbicides include others of the imidazolinone and sulfonylurea classes, such as primisulfuron, chlorsulfuron, imazamethabenz methyl, and triasulfuron. Other classes of AHAS herbicides known in the art include triazolopyrimidines, triazolopyrimidine sulfonamides, sulfamoylureas, sulfonylcarboxamides, sulfonamides, pyrimidyloxybenzoates, phthalides, pyrimidylsalicylates, carbamoylpyrazolines, sulfonylimino-triazinyl heteroazoles, N-protected valylanilides, sulfonylamide azines, pyrimidyl maleic acids, benzenesulfonyl carboxamides, substituted sulfonyldiamides, and ubiquinone-o.

As used in the specification and claims, the term “imidazolinone” means a herbicidal composition comprising one or more chemical compounds of the imidazolinone class, including by way of example and not limitation, 2-(2-imidazolin-2-yl)pyridines, 2-(2-imidazolin-2-yl)quinolines and 2-(2-imidazolin-2-yl) benzoates or derivatives thereof, including their optical isomers, diastereomers and/or tautomers exhibiting herbicidal activity, including by way of example and not limitation 2-[4,5-dihydro-4-methyl-4-(1-methylethyl)-5-oxo-1H-imidazol-2-yl]-3-quinolinecarboxylic acid (generic name imazaquin); 2-[4,5-dihydro-4-methyl-4-(1-methylethyl)-5-oxo-1H-imidazol-2-yl]-5-ethyl-3-pyridinecarboxylic acid (generic name imazethapyr); and 2-[4,5-dihydro-4-methyl-4-(1-methylethyl)-5-oxo-1H-imidazol-2-yl]-5-(methoxymethyl)-3-pyridinecarboxylic acid (generic name imazamox); 2-[4,5-dihydro-4-methyl-4-(1-methylethyl)-5-oxo-1H-imidazol-2-yl]-3-pyridinecarboxylic acid (generic name imazapyr); 2-[4,5-dihydro-4-methyl-4-(1-methylethyl)-5-oxo-1H-imidazol-2-yl]-5-methyl-3-pyridinecarboxylic acid) (generic name imazameth, also known as imazapic); and the other examples of imidazolinone herbicides given in the specification.

As used in the specification and claims, the term “sulfonylurea” means a herbicidal composition comprising one or more chemical compounds of the sulfonylurea class, which generally comprise a sulfonylurea bridge, —SO₂NHCONH—, linking two aromatic or heteroaromatic rings, including by way of example and not limitation 2-(((((4,6-dimethoxypyrimidin-2-yl)aminocarbonyl))aminosulfonyl))-N,N-dimethyl-3-pyridinecarboxamide (generic name nicosulfuron); 3-[4,6-bis (difluoromethoxy)-pyrimidin-2-yl]-1-(2-methoxycarbonylphenylsulfonyl) urea (generic name primisulfuron); 2-[[[[(4,6-dimethyl-2-pyrimidinyl)amino]carbonyl]amino]sulfonyl]benzoic acid methyl ester (generic name sulfometuron methyl); methyl 2-[[[[(4-methoxy-6-methyl-1,3,5-triazin-2-yl)amino]carbonyl]amino]sulfonyl]benzoate (generic name metsulfuron methyl); methyl-2-[[[[N-(4-methoxy-6-methyl-1,3,5-triazin-2-yl) methylamino]carbonyl]amino]sulfonyl]benzoate (generic name tribenuron methyl); methyl-3-[[[[(4-methoxy-6-methyl-1,3,5-triazin-2-yl)amino]carbonyl]amino]sulfonyl]-2-thiophenecarboxylate (generic name thifensulfuron methyl); 2-chloro-N-[(4-methoxy-6-methyl-1,3,5-triazin-2-yl)aminocarbonyl]benzenesulfonamide (generic name chlorsulfuron); ethyl 2-[[[[(4-chloro-6-methoxypyrimidin-2-yl)amino]carbonyl]amino]sulfonyl benzoate (generic name chlorimuron ethyl); methyl 2-[[[[N-(4-methoxy-6-methyl-1,3,5-triazin-2-yl)methylamino]carbonyl]amino]sulfonyl benzoate (generic name tribenuron methyl); 3-(6-methoxy-4-methyl-1,3,5-triazin-2-yl)-1-[2-(2-chloroethoxy)-phenylsulfonyl]-urea (generic name triasulfuron); and the other examples of sulfonylurea herbicides given in the specification.

As used in the specification and claims, unless otherwise clearly indicated by context, the term “plant” is intended to encompass plants at any stage of maturity, as well as any cells, tissues, or organs taken or derived from any such plant, including without limitation any embryos, seeds, leaves, stems, flowers, fruits, roots, tubers, single cells, gametes, anther cultures, callus cultures, suspension cultures, other tissue cultures, or protoplasts. Also, unless otherwise clearly indicated by context, the term “plant” is intended to refer to a photosynthetic organism or green plant including algae, mosses, ferns, gymnosperms, and angiosperms. The term excludes, however, both prokaryotes, and eukaryotes that do not carry out photosynthesis such as yeast, other fungi, and the so-called red plants and brown plants that do not carry out photosynthesis.

Unless otherwise clearly indicated by context, the “genome” of a plant refers to the entire DNA sequence content of the plant, including nuclear chromosomes, mitochondrial chromosomes, chloroplast chromosomes, plasmids, and other extra-nuclear or extra-chromosomal DNA. If, for example, a herbicide resistance nucleotide sequence is incorporated into the cells of a transformed plant in a plasmid or other genetic element that might not otherwise be consistently maintained and inherited by the plant and its progeny, then the herbicide resistance trait itself may be used to apply selective pressure upon such plants to maintain the herbicide resistance phenotype and genotype. Such a plant is considered to have the herbicide resistance nucleotide sequence in its “genome” within the contemplation of this definition.

Unless otherwise clearly indicated by context, the “progeny” of a plant includes a plant of any subsequent generation whose ancestry can be traced to that plant, e.g., F₁ progeny rice plants, F₂ progeny rice plants, F₃₀ progeny rice plants, varieties, hybrids, etc.

Unless otherwise clearly indicated by context, a “derivative” of a herbicide-resistant plant includes both the progeny of that herbicide-resistant plant, as the term “progeny” is defined above; and also any mutant, recombinant, or genetically-engineered derivative of that plant, whether of the same species or of a different species; where, in either case, the herbicide-resistance characteristics of the original herbicide-resistant plant have been transferred to the derivative plant. Thus a “derivative” of a Coreopsis plant with a resistant ALS enzyme would include, by way of example and not limitation, any of the following plants that express the same resistant ALS enzyme: F₁ progeny Coreopsis plants (including hybrids), F₂ progeny rice plants (including hybrids and backcrosses), F₃₀ progeny rice plants, etc., a transgenic maize plant transformed with a herbicide resistance nucleotide sequence from the resistant Coreopsis plant, and a transgenic sweet potato plant transformed with a herbicide resistance nucleotide sequence from the resistant Coreopsis plant.

The following definitions should be understood to apply throughout the specification and claims, unless otherwise clearly indicated by context.

An “isolated” nucleic acid sequence is an oligonucleotide sequence that is located outside a living cell. A cell comprising an “isolated” nucleic acid sequence is a cell that has been transformed with a nucleic acid sequence that at one time was located outside a living cell; or a cell that is the progeny of, or a derivative of, such a cell.

A “functional” or “normal” ALS enzyme is one that is capable of catalyzing the first step in the pathway for synthesis of the essential amino acids isoleucine, leucine, and valine; regardless of whether the enzyme expresses herbicide resistance.

A “resistant” plant is one that produces a functional ALS enzyme, and that is capable of reaching maturity when grown in the presence of normally inhibitory levels of a herbicide that normally inhibits ALS. The term “resistant” or “resistance,” as used herein, is also intended to encompass “tolerant” plants, i.e., those plants that phenotypically evidence adverse, but not lethal, reactions to one or more ALS herbicides. A “resistant” ALS enzyme is a functional ALS enzyme that retains substantially greater activity than does a wild-type ALS enzyme in the presence of normally inhibitory levels of an ALS herbicide, as measured by in vitro assays of the respective enzymes' activities. A “wild-type” or “sensitive” plant is one that produces a functional ALS enzyme, where the plant is sensitive to normally inhibitory levels of a herbicide that normally inhibits ALS. A “resistant” plant is a plant that is resistant to normally inhibitory levels of a herbicide that normally inhibits ALS (either due to a resistant ALS enzyme or another mechanism of resistance in the plant). Note that within the contemplation of this last definition, “wild-type” plants include cultivated varieties; the designation “wild-type” refers to the presence or absence of normal levels of herbicide sensitivity, and in the context of this specification and the claims the term “wild-type” carries no connotation as to whether a particular plant is the product of cultivation and artificial selection, or is found in nature in an uncultivated state.

A “wild-type” ALS enzyme or “wild-type” ALS sequence is an ALS enzyme or a DNA sequence encoding an ALS enzyme, respectively, that does not impart herbicide resistance. Thus, within the scope of this definition, a “wild-type” ALS may, for example, contain one or more mutations, provided that the mutations do not impart herbicide resistance. In some species, more than one wild-type ALS may naturally exist in different varieties. A “wild-type” ALS includes, for example, any of these multiple ALS enzymes from different varieties. A “wild-type” AHAS also includes, for example, a hybrid or mosaic of two or more of these wild-type AHAS enzymes.

The complete disclosures of all references cited in this specification are hereby incorporated by reference. Also incorporated by reference is the complete disclosure of N. Zhang et al., “Cloning of genes encoding acetolactate synthase from Coreopsis tinctoria Nutt. For resistance to sulfonylurea and imidazolinone herbicides” (manuscript 2006); N. Zhang et al., “Phylogenetic analysis of the acetolactate synthase gene from Coreopsis tinctoria” (manuscript poster 2006); N. Zhang et al., “Phylogenetic analysis of the acetolactate synthase (ALS gene from Coreopsis tinctoria,” Abstract, Plant & Animal Genomes XIV Conference (San Diego, Calif., Jan. 14-18, 2006). (Note: No presentation corresponding to this Abstract was in fact made at the Jan. 14-18, 2006 conference, nor otherwise publicly presented prior to the international filing date of the present application.) In the event of an otherwise irreconcilable conflict, however, the present specification shall control.

Notes on herbicide nomenclature—the following listing gives trade names, generic names, and chemical names for various herbicides: Pursuit™ or Newpath™ (imazethapyr: (±)-2-[4,5-dihydro-4-methyl-4-(1-methylethyl)-5-oxo-1H-imidazol-2-yl]-5-ethyl-3-pyridinecarboxylic acid); Scepter™ (imazaquin: 2-[4,5-dihydro-4-methyl-4-(1-methylethyl)-5-oxo-1H-imidazol-2-yl]-3-quinolinecarboxylic acid); Accent™ (nicosulfuron: 2-(((((4,6-dimethoxypyrimidin-2-yl)aminocarbonyl)) aminosulfonyl))-N,N-dimethyl-3-pyridinecarboxamide); Beacon™ (primisulfuron: 3-[4,6-bis(difluoromethoxy)-pyrimidin-2-yl]-1-(2-methoxycarbonylphenylsulfonyl)urea); Raptor™ (imazamox: (+)-5-methoxymethyl-2-(4-isopropyl-4-methyl-5-oxo-2-imidazolin-2-yl) nicotinic acid; Cadre™ (imazapic: (±)-2-[4,5-dihydro-4-methyl-4-(1-methyl-ethyl)-5-oxo-1H-imidazol-2-yl]-5-methyl-3-pyridinecarboxylic acid; alternate chemical name (±)-2-(4-isopropyl-4-methyl-5-oxo-2-imidazolin-2-yl)-5-methylnicotinic acid); Arsenal™ (imazapyr: 2-[4,5-dihydro-4-methyl-4-(1-methylethyl)-5-oxo-1H-imidazol-2-yl]-3-pyridinecarboxylic acid); Oust™ (sulfometuron methyl: chemical name 2-[[[[(4,6-dimethyl-2-pyrimidinyl)amino]carbonyl]amino]sulfonyl]benzoic acid methyl ester); Ally™ (metsulfuron methyl: methyl 2-[[[[(4-methoxy-6-methyl-1,3,5-triazin-2-yl)amino]carbonyl]amino]sulfonyl]benzoate); Harmony™ (mixture of thifensulfuron methyl and tribenuron methyl: mixture of methyl-3-[[[[(4-methoxy-6-methyl-1,3,5-triazin-2-yl)amino]carbonyl]amino]sulfonyl]-2-thiophenecarboxylate and methyl-2-[[[[N-(4-methoxy-6-methyl-1,3,5-triazin-2-yl) methylamino]carbonyl]amino]sulfonyl]benzoate); Pinnacle™ (thifensulfuron methyl: methyl-3-[[[[(4-methoxy-6-methyl-1,3,5-triazin-2-yl)amino]carbonyl]amino]sulfonyl]-2-thiophenecarboxylate); Glean™ or Telar™ (chlorsulfuron: 2-chloro-N-[(4-methoxy-6-methyl-1,3,5-triazin-2-yl)aminocarbonyl]benzenesulfonamide); Classic™ (chlorimuron ethyl: ethyl 2-[[[[(4-chloro-6-methoxypyrimidin-2-yl)amino]carbonyl]amino]sulfonyl benzoate); Express™ (tribenuron methyl: methyl 2-[[[[N-(4-methoxy-6-methyl-1,3,5-triazin-2-yl)methylamino]carbonyl]amino]sulfonyl benzoate); Assert™ (imazamethabenz methyl: m-toluic acid, 6-(4-isopropyl-4-methyl-5-oxo-2-imidazolin-2-yl)-, methyl ester; and p-toluic acid, 2-(4-isopropyl-4-methyl-5-oxo-2-imidazolin-2-yl)-, methyl ester); and Amber™ (triasulfuron: 3-(6-methoxy-4-methyl-1,3,5-triazin-2-yl)-1-[2-(2-chloroethoxy)-phenylsulfonyl]-urea); Staple™ (pyrithiobac sodium: sodium 2-chloro-6-[(4,6-dimethoxy pyrimidin-2-yl)thio]benzoate); and Matrix™ (rimsulfuron: N-((4,6-dimethoxypyrimidin-2-yl)aminocarbonyl)-3-(ethylsulfonyl)-2-pyridinesulfonamide).

1. An isolated nucleic acid sequence encoding a functional acetolactate synthase (ALS) having about 95% or greater amino acid sequence homology with a wild-type Coreopsis ALS; wherein the encoded ALS exhibits resistance to at least one herbicide that normally inhibits wild-type Coreopsis ALS; and wherein, as compared to the wild-type Coreopsis ALS, the encoded ALS has amino acid substitutions at both amino acid positions Pro197 and Trp574.

2. (canceled)

3. A nucleic acid sequence as recited in claim 1, wherein the encoded ALS is identical to a wild-type ALS, except for the amino acid substitutions at positions Pro197 and Trp574.

4. A nucleic acid sequence as recited in claim 1, wherein, as compared to the wild-type Coreopsis ALS, the encoded ALS has amino acid substitutions at amino acid position Glu322, or at position Gly476, or at both positions Glu322 and Gly476.

5. A nucleic acid sequence as recited in claim 1, wherein the herbicide is selected from the group consisting of herbicidally effective imidazolinones, sulfonylureas, triazolopyrimidines, pyrimidyloxybenzoates, and phthalide compounds.

6. A nucleic acid sequence as recited in claim 1, wherein the herbicide is selected from the group consisting of imazapyr, chlorsulfuron, and sulfometuron methyl, and derivatives of these herbicides.

7. A transformation vector comprising a nucleic acid sequence as recited in claim 1.

8. A host plant cell comprising a nucleic acid sequence as recited in claim 1.

9. A nucleic acid construct comprising a nucleic acid sequence as recited in claim 1, wherein said sequence is operably linked to a promoter that is functional in plants.

10. A nucleic acid construct as recited in claim 9, wherein said promoter is a native plant ALS promoter.

11. A nucleic acid construct as recited in claim 9, wherein said promoter is a native Coreopsis ALS promoter.

12. A nucleic acid construct as recited in claim 9, wherein said promoter is the cauliflower mosaic virus 35S promoter.

13. A method of conferring resistance to ALS-inhibiting herbicides to a plant, comprising incorporating a nucleic acid construct as recited in claim 9 into the genome of a plant.

14. A plant transformed with a nucleic acid construct as recited in claim 9.

15. A process for controlling weeds in the vicinity of a plant as recited in claim 14, said process comprising applying a herbicide to the weeds and to the plant, wherein the herbicide normally inhibits acetohydroxyacid synthase, at levels of the herbicide that would normally inhibit the growth of a plant of the same species.

16. An isolated nucleic acid sequence encoding a functional acetolactate synthase (ALS) identical to a herbicide-resistant ALS expressed by a Coreopsis plant with ATCC accession number PTA-3981.

17. (canceled)

18. A transformation vector comprising a nucleic acid sequence as recited in claim 16.

19. A host plant cell comprising a nucleic acid sequence as recited in claim 16.

20. A nucleic acid construct comprising a nucleic acid sequence as recited in claim 16, wherein said sequence is operably linked to a promoter that is functional in plants.

21. A nucleic acid construct as recited in claim 20, wherein said promoter is a native plant ALS promoter.

22. A nucleic acid construct as recited in claim 20, wherein said promoter is a native Coreopsis ALS promoter.

23. A nucleic acid construct as recited in claim 20, wherein said promoter is the cauliflower mosaic virus 35S promoter.

24. A method of conferring resistance to ALS-inhibiting herbicides to a plant, comprising incorporating a nucleic acid construct as recited in claim 20 into the genome of a plant.

25. A plant transformed with a nucleic acid construct as recited in claim 20.

26. A process for controlling weeds in the vicinity of a plant as recited in claim 25, said process comprising applying a herbicide to the weeds and to the plant, wherein the herbicide normally inhibits acetohydroxyacid synthase, at levels of the herbicide that would normally inhibit the growth of a plant of the same species.

27. An isolated nucleic acid sequence encoding a functional acetohydroxyacid synthase (ALS) having about 95% or greater amino acid sequence homology with the wild-type ALS from a green plant; wherein the encoded ALS exhibits resistance to at least one herbicide that normally interferes with the wild-type ALS; and wherein, as compared to the wild-type ALS for plants of the same species, the encoded ALS has amino acid substitutions at both amino acid positions Pro197 and Trp574.

28. (canceled)

29. A nucleic acid sequence as recited in claim 27, wherein the encoded ALS is identical to a wild-type ALS, except for the amino acid substitutions at positions Pro197 and Trp574.

30. A nucleic acid sequence as recited in claim 27, wherein, as compared to the wild-type Coreopsis ALS, the encoded ALS has amino acid substitutions at amino acid position Glu322, or at position Gly476, or at both positions Glu322 and Gly476.

31. A nucleic acid sequence as recited in claim 27, wherein the herbicide is selected from the group consisting of herbicidally effective imidazolinones, sulfonylureas, triazolopyrimidines, pyrimidyloxybenzoates, and phthalide compounds.

32. A nucleic acid sequence as recited in claim 27, wherein the herbicide is selected from the group consisting of imazapyr, chlorsulfuron, and sulfometuron methyl, and derivatives of these herbicides.

33. A transformation vector comprising a nucleic acid sequence as recited in claim 27.

34. A host plant cell comprising a nucleic acid sequence as recited in claim 27.

35. A nucleic acid construct comprising a nucleic acid sequence as recited in claim 27, wherein said sequence is operably linked to a promoter that is functional in plants.

36. A nucleic acid construct as recited in claim 35, wherein said promoter is a native plant ALS promoter.

37. A nucleic acid construct as recited in claim 35, wherein said promoter is the cauliflower mosaic virus 35S promoter.

38. A method of conferring resistance to an ALS-inhibiting herbicide to a plant, comprising incorporating a nucleic acid construct as recited in claim 35 into the genome of a plant.

39. A method as recited in claim 38, wherein the nucleic acid construct is incorporated into the genome of the plant by site-directed mutagenesis.

40. A transformed plant produced by the method of claim 39.

41. A plant transformed with a nucleic acid construct as recited in claim 35.

42. A process for controlling weeds in the vicinity of a plant as recited in claim 41, said process comprising applying a herbicide to the weeds and to the plant, wherein the herbicide normally inhibits acetohydroxyacid synthase, at levels of the herbicide that would normally inhibit the growth of a plant of the same species.

43. An isolated nucleic acid sequence comprising a sequence selected from the group consisting of SEQ ID NOS 13, 14, 15, and 16; or comprising a sequence that encodes the same amino acid sequence as one of these nucleic acid sequences, in accordance with the degeneracy of the genetic code.

44. A nucleic acid sequence as recited in claim 43, wherein said nucleic acid sequence comprises a sequence selected from the group consisting of SEQ ID NOS 13, 14, 15, and 16.

45. A nucleic acid sequence as recited in claim 43, wherein said nucleic acid sequence comprises SEQ ID NO 13.

46. A nucleic acid sequence as recited in claim 43, wherein said nucleic acid sequence comprises SEQ ID NO 14.

47. A nucleic acid sequence as recited in claim 43, wherein said nucleic acid sequence comprises SEQ ID NO 15.

48. A nucleic acid sequence as recited in claim 43, wherein said nucleic acid sequence comprises SEQ ID NO 16.

49. A transformation vector comprising a nucleic acid sequence as recited in claim 43.

50. A host plant cell comprising a nucleic acid sequence as recited in claim 43.

51. A nucleic acid construct comprising a nucleic acid sequence as recited in claim 43, wherein said sequence is operably linked to a promoter that is functional in plants.

52. A nucleic acid construct as recited in claim 51, wherein said promoter is a native plant ALS promoter.

53. A nucleic acid construct as recited in claim 51, wherein said promoter is the cauliflower mosaic virus 35S promoter.

54. A method of conferring resistance to an ALS-inhibiting herbicide to a plant, comprising incorporating a nucleic acid construct as recited in claim 51 into the genome of a plant.

55. A plant transformed with a nucleic acid construct as recited in claim 51.

56. A plant transformed with a nucleic acid construct as recited in claim 51 by site-directed mutagenesis.

57. A process for controlling weeds in the vicinity of a plant as recited in claim 55, said process comprising applying a herbicide to the weeds and to the plant, wherein the herbicide normally inhibits acetohydroxyacid synthase, at levels of the herbicide that would normally inhibit the growth of a plant of the same species.

58-71. (canceled)


SEQUENCE LISTING (28 sequences)

SEQ ID NO: 1 - DNA, 21 bp, Artificial sequence
  Synthetic Primer
ttggaacagatgcgtttcaag

SEQ ID NO: 2 - DNA, 23 bp, Artificial sequence
  Synthetic primer
ctccaactgaaccaccataccca

SEQ ID NO: 3 - DNA, 19 bp, Artificial sequence
  Synthetic primer
tcgcataaaccgtcccgtg

SEQ ID NO: 4 - DNA, 22 bp, Artificial sequence
  Synthetic primer
atcactcgaagccgggaacgct

SEQ ID NO: 5 - DNA, 19 bp, Artificial sequence
  Synthetic primer
tcatcccatttcgggacca

SEQ ID NO: 6 - DNA, 22 bp, Artificial sequence
  Synthetic primer
agataacccggtaacctcatcg

SEQ ID NO: 7 - DNA, 24 bp, Artificial sequence
  Synthetic primer
gatcattctccgcgggacttgacc

SEQ ID NO: 8 - DNA, 20 bp, Artificial sequence
  Synthetic primer
ggccggaagtggctatgcac

SEQ ID NO: 9 - DNA, 18 bp, Artificial sequence
  Synthetic primer
atggcggctgtcactccc

SEQ ID NO: 10 - DNA, 23 bp, Artificial sequence
  Synthetic primer
ccaaccctagacaccattcaaac

SEQ ID NO: 11 - DNA, 25 bp, Artificial sequence
  Synthetic primer
ccccgtacataacactctatatgcg

SEQ ID NO: 12 - DNA, 1381 bp, Coreopsis tinctoria
cacagaagtatgccaagcgaggggggggggtccaattatgcttgggcttgatttcatagccgaagaactgcagcaagtcgaggatccagttaagcttgggctcaatttcatcgacgaagaagaaggaagaaccgtagaaactgcaatcagaagtgtcgatggtaatttggtagttttgaacgagggatcgaggggtagtttggtacttgtatcttggattggatggaataattaactctaatttaacagagggacacgtactgattaaattttaaaccacgaggacgcaaattattgggtagttaaaacttcaatgtattttgtacatataatttccttcaaattttttttacatataactcattgttcactgtatgcatgctttgtttgtttgtaccgtatgctttcattatttctacttctctttgttcctttagtttacacgtcgtatgcagacgacaacgactcgtcgtttcgtatcaaccgtcggttcgccagcgacaaagaccaacaccggatttacaactccctactagtgttcatatatgttgtttagggattccaaattattatataatagctatggttgtattaaccattcaactaaaaaaacaattaatctcttcgattgctattgtatatacatgatttcttgtaattcaaagtcctataagtatcattttaggattacatcattgattagagcattaaattatctgtgtatatgaacacaattcacatacataataaattgaaaaaaaaaagtatcttacaaaaacacatgattataaaaatgttaaatttataatttactcggtccctacatatttaacactcagttgatcagattataattttcaaaagacgtgtacttgatttgagtattatgtttttatcctttcattaaataatgtaagatagagtttggtgagttaatatacctattggtttttggaagttcatagcctaatcacaaaaagaaaagggactaaacttgcatctttcaacctctaattttttttttttttttttttttaacagccaaagggatcactgacgggccactgacccacccaatcatatccattccaccctcaggaggaaacccaccattaaccaatagatgagaaaaccccctggcccaacttgagccccctattttttgtctcaagtgagacttgaactcacaaccttatgattgtaagccacacccctttgccactagactaaacgagtggtgtaacctttaaattagacagacacctaaaaacactggaactcaaaacaaaaatcaaccacatgtttggttttcctgaccacattcattcctatattattaaccagaaaaacaccacaaaccaaccctagacaccattcaaac

SEQ ID NO: 13 - DNA, 2104 bp, Coreopsis tinctoria
atggcggctgtcactccctcaaaccctcccatctccacctatccctcaccgccgtccgccgccgccaccaccaccacaaaccgccccaccctccttcctcgtctcacattctcagcctcctccactctcccaaaacgacaccgtctcaccatcgtgaatgtcctctccaactcgaaacccaccaccacaaccaccactcattcaccccctccgtttcccgctcggttcgcccctgaccagccccgtaaaggctccgacgtcctcgtagaagccctcgaacgcgaaggcgtcaccgacgtcttcgcctacccgggtggcgcctctatggagatccaccaagccctaacccggtccaaaatcatccgtaacgtcctcccgcgccacgagcaaggcggcgtcttcgcggccgagggctacgcccgggcctcgggtttacccggcgtgtgcatagccacttccggccccggagctaccaatttggttagcggtcttgcggatgcattacttgatagtgtccccatggttgcgattacgggtcaagtcccgcggagaatgatcgggaccgatgcgtttcaagaaaccccgattgttgaggtaacacggtctattactaaacataattaccttgtgttagatgtcgaggatattcctagaattgttagggaggctttttatcttgcgagatcgggtcggcccggcccggttttgattgatatcccgaaagatatacaacaacagttagtggtcccgaaatgggatgaaccgatgaggttaccgggttatcttgctaggttaccgaaaacacaaagtgatgataatagtttagatcaaattgttcggttgatatcggaagcgaagagaccggttttgtatgtgggtggtggttgtttgaattcgggtgaggaattgggaaggtttgtcgagcttacggggatcccggttgctagtactttaatgggtcttggagcgttcccggcttcgagtgatttatcgcttcatatgcttggaatgcacgggacggtttatgcgaattacgcggttgataagagtgatttgttgcttgcgttcggggtgaggtttgatgatcgtgtgacgggaaagctcgaggcgtttgctagccgagctaaaatcgtgcatatcgatatcgattcggctgaaatcgggaagaataagcagcctcacgtgtcggtttgtggtgatattaaggtcgcattacagggcctcaacaagattttggagggaaaaggttcggttactaatcttgattttgcgaattggaggaaggagttggacgagcaaaaggtgaagtttccgttaagttttaaaacgtttggtgaagcgattccgccacaatacgcgattcaggttcttgacgagttaacgggcggtaatgcgattattagcaccggggtcgggcaacatcagatgtgggctgcccagttttacaagtacaacaaaccgagacagtggttgacttcgggtggactcggggcgatgggtttcgggctgcccgctgctatcggggcagctgttgcgagacccgacgcggtagtggtggatatcgatggtgacgggagctttataatgaatgttcaagaattagcgacaatccgcgtcgaaaatctgccggttaagatattattacttaataatcagcatttgggtatggtggttcaattggaggaccgcttttacaaagcgaatcgggctcatacttacttaggaaacccgtcaaacgagactgaaatattcccgaacatgttgaagtttgcggaagcgtgtggtatcccggctgcccgagtgacccggaaggcggacgtacgggcagctattcagaaaatgttggatacgccggggccgtacttgttggatgtaattgtgccacatcaagaacatgttttgcccatgattccagctggcggcggtttcgcggatgtgatcactgaaggtgatggcagaataaaatactgaggtttgaagtcgcatatagagtgttatgtaagttgtttgttttttaagttaatgttttgttgtgtaatttagtttgaatgttctggttcggtttatcaacgtttcttttaataaaaaaaaaaaaaaaaaaaaa

SEQ ID NO: 14 - DNA, 2135 bp, Coreopsis tinctoria
atggcggctgtcactccctcaaaccctcccatctccacctatccctcaccgccgtccgccgccgccaccaccaccacattccgccccaccctccttcctcatctcacattctcagcctcctccactctccccaaacgacaccgtctcaccatcgtgaatgtcctctccaactcgaaacccaccaccacaaccaccactcattcaccccctccgtttcccgctcggttcgcccctgaccagccccgtaaaggctccgacgtcctcgtcgaagccctcgaacgcgaaggcgtcaccgacgtcttcgcctacccgggtggcgcctctatggagatccaccaagccctaacccggtccaaaatcatccgaaacgtcctcccgcgtcacgagcaaggcggcgtcttcgcggccgagggctacgcccgcgcctcgggcttacccggcgtgtgcatagccacttccggccccggagctaccaatttggttagcggtcttgcggatgcattacttgatagtgtccccatggtcgcgattacgggtcaagtcctgcggagaatgatcgggactgatgcgtttcaagaaaccccgattgttgaggtaacacggtctattactaaacataattaccttgtgttagatgtcgaggatattcctagaattgttagggaggctttttatcttgcgagatcgggtcggcccggcccggttttgattgatatcccgaaagatatacaacaacagttagtggtcccgaaatgggatgaaccgatgaggttaccgggttatcttgctaggttaccgaaaacacaaagtgatgataatagtttagatcaaattgttcggttgatatcggaagcgaagagaccggttttgtatgtgggtggtggttgtttgaattcgggtgaggaattgggaaggtttgtcgagcttacggggatcccggttgctagtactttaatgggtcttggagcgttcccggcttcgagtgatttatcgcttcatatgcttggaatgcacgggacggtttatgcgaattacgcggttgataagagtgatttgttgcttgcgttcggggtgaggtttgatgatcgtgtgacgggaaagctcgaggcgtttgctagccgagctaaaatcgtgcatatcgatatcgattcggctgaaatcgggaagaataagcagcctcacgtgtcggtttgtggtgatattaaggtcgcattacagggcctcaacaagattttggagggaaaaggttcggttactaatcttgattttgcgaattggaggaaggagttggacgagcaaaaggtgaagtttccgttaagttttaaaacgtttggtgaagcgattccgccacaatacgcgattcaggttcttgacgagttaacgggcggtaatgcgattattagcaccggggtcgggcaacatcagatgtgggctgcccagttttacaagtacaacaaaccgagacagtggttgacttcgggtggactcggggcgatgggtttcgggctgcccgctgctatcggggcagctgttgcgagacccgacgcggtagtggtggatatcgatggtgacgggagctttataatgaatgttcaagaattagcgacaatccgcgtcgaaaatctgccggttaagatattattacttaataatcagcatttgggtatggtggttcaattggaggaccgcttttacaaagcgaatcgggctcatacttacttaggaaacccgtcaaacgagactgaaatattcccgaacatgttgaagtttgcggaagcgtgtggtatcccggctgcccgagtgacccggaaggcggacgtacgggcagctattcagaaaatgttggatacgccggggccgtacttgttggatgtaattgtgccacatcaagaacatgttttgcccatgattccagctggcggcggtttcgcggatgtgatcactgaaggtgatggcagaataaaatactgaggtttgaagtcgcatatagagtgttatgtaagttgttttttttttaagttgttttgttgtgtaatttagtttgaatgttctcgttcagtttatcaacgttttctttagtacctttctcgaataaatttccgtcttttatctaaaaaaaaaaaaaaaaaaaaaaa

SEQ ID NO: 15 - DNA, 2242 bp, Coreopsis tinctoria
atggcggctgtcactccctcaagctctcccatctccacctatccctcaccgccgtccgccgccgccaccaccaccacattccgccccaccctccttcctcatctcacattctcagcctcctccactctcccaaaacgacaccgtctcaccatcgtgaatgtcctctccaactcgaaacccaccaccacaaccaccacgcattcaccccctccgtttcccgctcggttcgcccctgaccagccgcgtaaaggctccgacgtcctcgtcgaagccctcgaacgcgaaggcgtcaccgacgtcttcgcctacccgggtggcgcctctatggagatccaccaagccctaacccggtccaaaatcatccgaaacgtcctcccgcgtcacgagcaaggcggcgtcttcgcggccgaggggtacgcccgggcctcgggtttacccggcgtgtgcatagccacttccggccccggagctaccaatttggttagcggtcttgcggatgcattacttgatagtgttcccatggtcgcaattacgggtcaagtcccgcggagaatgatcgggaccgatgcgtttcaagaaaccccgattgttgaggtaacacggtctattactaaacataattaccttgtgttagatgtcgaggatattcctagaattgttagggaggctttttatcttgcgagatcgggtcggccgggcccggttttgattgatatcccgaaagatatacaacaacaattagtggttccgaaatgggatgaaccgatgaggttgccgggttatcttgctaggttaccgaaaacacaaagtgatgataatagtttagagcagattgttcggttgatttcggaagcgaaaagaccggttttgtatgtgggtggcggttgtttgaactcgggcgacgagttggggaggtttgtcgagcttacggggattccggttgctagtactttaatgggtcttggagcgtacccggcttccagtgatttatcgcttcatatgcttggaatgcacggaacggtttatgcgaattacgcggttgataagagtgatttgttgcttgcgtttggggtgaggttcgatgatcgtgtgacggggaagctcgaggcgtttgctagccgagctaaaatcgtgcatatcgatatcgattcggctgaaatcgggaagaataagcagcctcacgtgtcggtttgtggtgatattaaggtcgcgttacaaggtcttaacaagattttggagggaaaaggttcggttactaatctcgattttgcgaattggaggaaggagttggacgagcaaaaggtgaagtttccgttaagttttaaaacgtttggtgaagcgattccgccacaatacgcgattcaggttcttgacgagttaacgggcggtaatgcgattattagcaccggggtcgggcaacatcagatgtgggctgcccagttttacaagtacaacaaaccgagacagtggttgacttctggtggactcggggcgatgggtttcgggctgcccgctgctatcggggcagctgttgcgagaccggatgcggtggtggttgatatcgatggcgacgggagctttataatgaacgttcaagaattagcgacaatccgcgtcgaaaatctgccggttaagatattattacttaataatcagcatttgggtatggtggttcaatgggaggaccggttttacaaagcgaatcgggcgcatacctacttaggaaacccgtcaaacgagactgaaatattcccgaacatgttgaagtttgcggaagcgtgtggtatcccggctgcccgagtgaccaggaaggcggacgttcgggcggctattcagaagatgttggatacgccggggccgtacttgttggatgtaattgtgccacatcaagaacatgttttgcccatgattccagctggcggcggtttcgcggatgtgatcactgaaggtgatggcagaattaaatactgaggtttgaagtcgcatatagagtgttatttaagttgtttttttttaagttgttttgttgtgtaatttagtttgaatgttctcgttcagtttatcaacgttttctttagtacctttctcgaataaatttccgtcttttatcttttggtttatggttaggctttatattatagaccaaattatgtttttattaagtgtttatatgttgttttctataagtccaaaagaatagttagttgagaatgtgtttggtgaaaaaaaaaaaaaaaaaaaa

SEQ ID NO: 16 - DNA, 2138 bp, Coreopsis tinctoria
atggcggctgtcactccctcaaaccctcccatctccacctatccctcaccgccgtccgccgccgccaccaccaccacattccgccccaccctccttcctcatctcacattctcagcctcctccactctccccaaacgacaccgtctcaccatcgtgaatgtcctctccaactcgaaacccaccaccacaaccaccactcattcaccccctccgtttcccgctcggttcgcccctgaccagccccgtaaaggctccgacgtcctcgtcgaagccctcgaacgcgaaggcgtcaccgacgtcttcgcctacccgggtggcgcctctatggagatccaccaagccctaacccggtccaaaatcatccgaaacgtcctcccgcgtcacgagcaaggcggcgtcttcgcggccgagggctacgcccgcgcctcgggcttacccggcgtgtgcatagccacttccggccccggagctaccaatttggttagcggtcttgcggatgcattacttgatagtgtccccatggtcgcgattacgggtcaagtcctgcggagaatgatcgggactgatgcgtttcaagaaaccccgattgttgaggtaacacggtctattactaaacataattaccttgtgttagatgttgaggatattcctaggattgttagggaggccttttatcttgcgagatcgggtcggcccggcccggttttgattgatatcccgaaagatatacaacaacaattagtggtccctaaatgggatgaaccgatgaggttaccgggttatcttgctaggttaccgaaaacacaaagtgatgataatagtttagagcaaattgttcggttgatatcggaagcgaagagaccggttttgtatgtgggtggcggttgtttgaattcgggtgacgagttggggaggtttgtcgatcttacggggatcccggttgctagtactttaatgggtcttggagcgtacccggcttcgagtgatttatcgcttcatatgcttggaatgcacgggacggtttatgcgaattacgcggttgataagagtgatttgttgcttgcgttcggggtgcggtttgatgatcgagtgacggggaagctcgaggcgtttgctagccgagctaaaatcgtgcacattgatatcgattcggctgaaatcgggaagaataagcagcctcacgtgtcggtttgtggtgatattaaggtcgcgttacagggtctcaacaagattttggagggaaaaggttcggttactaatcttgattttgcgaattggaggaaggagttggacgagcaaaaagtgaagtttccgttaagttttaaaacgtttggtgaagcgattccgccacaatacgcgattcaggttcttgacgagttaacgtgcggtaatgcgattattagcaccggggtcgggcaacatcagatgtgggctgcccagttttacaagtacaacaaaccgagacagtggttgacttcgggtggactcggggcgatgggtttcgggctgcccgctgctatcggggcagctgttgcgaggccggatgcggtggtggttgatatcgatggcgacgggagctttataatgaatgttcaagaattagcgacaatccgcgtcgaaaatctgccggttaagatattattacttaataatcagcatttgggtatggtggttcaatgggaggatcggttttacaaagcgaatcgggcgcatacttatttaggaaacccgtcaaacgagacggaaatattcccgaacatgttgaagtttgcggaagcgtgtggtatcccggctgcccgagtgacccggaaggcggatgttcgggcagcaattcagaagatgttggatacgccggggccgtacttgttggatgttattgtgccgcatcaagaacatgttttacccatgattccagctggcggcggtttcgcggatgtgatcactgaaggtgatggcagaataaaatactgaggtttgaggtcgcatatagagtgttatgtaagttgtttgttttttaagttgatgttttgttgtgtaatttagtttgaatgttctcgttcagtttatcaacgttttctttaatacctttctcgaataaatttccttcttttaaaaaaaaaaaaaaaaaaaaaaaaaaa

SEQ ID NO: 17 - protein, 656 aa, Coreopsis tinctoria
MAAVTPSNPPISTYPSPPSAAATTTTNRPTLLPRLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLARSGRPGPVLIDIPKDIQQQLVVPKWDEPMRLPGYLARLPKTQSDDNSLDQIVRLISEAKRPVLYVGGGCLNSGEELGRFVELTGIPVASTLMGLGAFPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQLEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 18 - protein, 656 aa, Coreopsis tinctoria
MAAVTPSNPPISTYPSPPSAAATTTTFRPTLLPHLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVLRRMIGTDAFQETPIVEVTRSITKHNYLVLDVEDIPRIVREAFYLARSGRPGPVLIDIPKDIQQQLVVPKWDEPMRLPGYLARLPKTQSDDNSLDQIVRLISEAKRPVLYVGGGCLNSGEELGRFVELTGIPVASTLMGLGAFPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQLEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 19 - protein, 656 aa, Coreopsis tinctoria
MAAVTPSSSPISTYPSPPSAAATTTTFRPTLLPHLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLARSGRPGPVLIDIPKDIQQQLVVPKWDEPMRLPGYLARLPKTQSDDNSLEQIVRLISEAKRPVLYVGGGCLNSGDELGRFVELTGIPVASTLMGLGAYPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 20 - protein, 656 aa, Coreopsis tinctoria
MAAVTPSNPPISTYPSPPSAAATTTTFRPTLLPHLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVLRRMIGTDAFQETPIVEVTRSITKHNYLVLDVEDIPRIVREAFYLARSGRPGPVLIDIPKDIQQQLVVPKWDEPMRLPGYLARLPKTQSDDNSLEQIVRLISEAKRPVLYVGGGCLNSGDELGRFVDLTGIPVASTLMGLGAYPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTCGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 21 - DNA, 1981 bp, Coreopsis tinctoria
atggcggctgtcactccctcaaaccctcccatctccacctatccctcaccgccgtccgccgccgccaccaccaccacaaaccgccccaccctccttcctcgtctcacattctcagcctcctccactctcccaaaacgacaccgtctcaccatcgtgaatgtcctctccaactcgaaacccaccaccacaaccaccactcattcaccccctccgtttcccgctcggttcgcccctgaccagcctcgtaaaggctccgacgtcctcgttgaagccctcgaacgcgaaggcgtcaccgacgtcttcgcctacccgggtggcgcctctatggagatccaccaagccctaacccggtccaaaatcatccgtaacgtcctcccgcgtcacgagcaaggcggcgtcttcgcggccgagggctacgcccgggcctcgggtttacccggcgtgtgcatagccacttccggccccggagctaccaatttggttagcggtcttgcggatgcattacttgatagtgtccccatggtcgcaattacgggtcaagtcccgcggagaatgatcgggactgatgcgtttcaagaaaccccgattgttgaggtaacacggtctattactaaacataattaccttgtgttggatgtcgaggatattcctagaattgttagggaggccttttatcttgcgagatcgggtcggcccggcccggttttgattgatatcccgaaagatatacaacaacagttagtggtcccgaaatgggatgaaccgatgaggttaccgggttatcttgctaggttaccgaaaacacaaagtgatgataatagtttagatcaaattgttcggttgatatcggaagcgaagagaccggttttgtatgtgggcggcggttgtttgaattccggtgacgagttggggaggtttgtcgagcttacgggtatcccggttgctagtactttaatgggtcttggagcgttcccggcttcgagtgatttatcgcttcatatgcttggaatgcacgggacggtttatgcgaattacgcggttgataagagtgatttgttgcttgcgttcggggtgaggtttgatgatcgagtgacggggaagctcgaggcgtttgctagccgagctaaaatcgtgcacattgatatcgattcggctgaaatcgggaagaataagcagcctcatgtgtcggtttgtggtgatattaaggtcgcattacagggtctcaacaagattttggagggaaaaggttcggttactaatctcgattttgcgaattggaggaaggagttggacgagcaaaaggtgaagtttccgttaagtttcaaaacgtttggtgaagcgattccgccgcaatacgcgattcaggttcttgacgagttaacgggcggtaatgcgattatcagcaccggggtcgggcagcatcagatgtgggctgcccagttttacaagtacaacaaaccgagacagtggttgacttcgggtggactcggggcgatgggtttcgggctgcccgctgctatcggggcagctgttgcgagacccgacgcggtagtggtggatatcgatggtgacgggagctttataatgaatgttcaagaattagcgacaatccgtgtcgaaaatctgccggttaagatattattacttaataatcagcatttgggtatggtggttcaatgggaggaccggttttacaaagcgaaccgggcgcatacttacttaggaaacccgtcaaacgagactgaaatattcccgaacatgttgaagtttgcggaagcgtgtggtatcccggctgcccgagtgacccggaaggcggatgttcgggcagctattcagaagatgttggatacaccggggccgtacttgttggatgttatcgtgccacatcaagaacatgttttacccatgattccagctggcggcggtttcgcggatgtgatcactgaaggtgatggcagaataaaatactgaggtttgaagt

SEQ ID NO: 22 - DNA, 1984 bp, Coreopsis tinctoria
atggcggctgtcactgactcaaaccctcccttctccacctatccctcaccgccgtccgccgcggccaccaccaccaccacattccgccccaccctccttcctcatctcacattctcagcctcctccactctcccaaaacgacaccgtctcaccatcgtgaatgtcctctccaactccaaacccaccaccacaaccacctgtcattcacccccgccgtttcccgctcggttcgcccctgaccagccgcgtaaaggctccgacgtcctcatcgatcccctcaaaggccaaggcgtccccgacgtctttgcctacccgggtggcgcctctatggagatccaccaagccctaacccggtccaaaatcatccgaaacgtcctcccgcgtcacgagcaaggtggagtcttcgcggccgagggctacccccgcgcctcgggcttacccggcgtgtgcatacccacttccggccccggaactaccaatttggttagcggtcgtgcggatgcgttacttgagagtgtccgcatggtcgcaattacaggtcaagtcccgccaagaatgatcggcaccgatgcgtttcaagaaaccccgattgttgaggtaacacggtctattactaaacataattaccttgtgttagatgtcgaggatattcctagaattgttagggaggccttttatcttgcgaggtcgggtcggcccggcccggttttgattgatatcccgaaagacatacaacaacaattagtggtaccgaaatgggacgaaccgatgaggttaccgggttatcttgctaggttaccgaaaacacaaagtgatgataatagtttagatcaaattgttcggttgatatcggaagcgaaaagaccggttttgtatgtgggtggtggttgtttgaattccggtgacgagttggggaggtttgtcgagcttacggggattccggttgctagtactttaatgggtcttggagcgttcccggcttcgagtgatttatcgcttcatatgcttggaatgcacgggacggtttatgcgaattacgcggtcgataagagtgatttattgcttgcgtttggggtgcggtttgatgatcgtgtgacgggaaagctcgaggcgtttgctagccgagctaaaatagtgcatatcgatatcgattcggctgaaatcgggaagaataagcagcctcacgtgtcggtttgtggtgatataaaggtcgcgttacagggtctcaacaagattttggagggaaaagattcggttactaatctcgattttgcgaattggaggaaggagttggacgagcaaaaggtgaagtttccgttaagttttaaaacgtttggtgaagcgattccgccacaatacgcgattcaggttcttgacgagttaacgggcggtaatgcgattattagcaccggggtcgggcaacatcagatgtgggctgcccagttttacaagtacaacaaaccgagacagtggttgacttctggtggactcggggcgatgggtttcgggctgcccgctgctatcggggcagccgttgcgagacccgacgcggtagtggtggacatcgatggcgacgggagctttataatgaacgttcaagaattagcgacaatccgcgtcgaaaatctgccggttaagatattattacttaataatcaacatttgggtatggtggttcaatgggaggatcggttttacaaagcgaatcgggcgcatacttacttaggaaacccgtcaaacgagacggaaatattcccgaacatgttgaagtttgcggaagcgtgtggtatcccggctgcccgagtgacccggaaggccgacgttcgggcagcaattcagaaaatgttggatacgcctgggccgtatttgttggatgttattgtgccacatcaagaacatgttttgcccatgattccagctggcggtggtttcgcggatgtgatcactgaaggtgatggcagaataaaatactgaggtttgaagt

SEQ ID NO: 23 - DNA, 1981 bp, Coreopsis tinctoria
atggcggctgtcactccctcaaaccctcccatctccacctatccctcaccgccgtccgccgccgccaccaccaccacaaaccgccccaccctccttcctcgtctcacattctcagcctcctccactctcccaaaacgacaccgtctcaccatcgtgaatgttctctccaactcgaaacccaccaccacaaccaccactcattcaccccctccgtttcccgctcggttcgcccctgaccagccgcgtaaaggctccgacgtcctcgtagaagccctcgaacgcgaaggcgtcaccgacgtcttcgcctacccgggtggcgcctctatggagatccaccaagccctaacccggtccaaaatcatccgtaacgtcctcccgcgtcacgagcaaggcggcgtcttcgcggccgagggctacgcccgggcctcgggtttacccggcgtgtgcatagccacttccggccccggagctaccaatttggttagcggtcttgcggatgcattacttgatagtgtccccatggtcgcaattacgggtcaagtcccgcggagaatgatcgggaccgatgcgtttcaagaaaccccgattgttgaggtaacacggtctattactaaacataattaccttgtgttagatgtcgaggatattcctagaattgttagggaagccttttatcttgcgaggtcgggtcggcccggcccggttttgattgatatcccgaaagatatacaacaacagttagtggtgccgaaatgggatgaaccgatgaggttaccgggttatcttgctaggttaccgaaaacacaaagtgatgataatagtttagagcagattgttcggttgatatcggaagctaagagaccggttttgtatgtgggtggcggttgtttgaattcgggtgaggagttggggaggtttgtcgagcttacggggatcccggttgctagtactttaatgggtcttggagcgtacccggcttcgagtgatttatcgcttcatatgcttggaatgcacgggacggtttatgcgaattacgcggttgataaaagtgatttgttgcttgcgttcggggtgcggtttgatgatcgtgtgacgggaaagctcgaggcgtttgctagccgagctaaaatcgtgcacattgatatcgattcggctgaaatcgggaagaataagcagcctcacgtgtcagtttgtggtgatattaaggttgcgttacagggtctcaacaagattttggagggaaaaggttcggttactaatctcgattttgcgaattggaggaaggagttggacgagcaaaaggtgaagtttccgttaagttttaaaacgtttggtgaagcgattccgccacaatacgcgattcaggttcttgacgagttaacgggcggtaatgcgattattagcaccggggtcgggcaacatcagatgtgggctgcccagttttacaagtacaacaaaccgagacagtggttgacttcaggtggactcggggcgatgggtttcgggctgcccgctgctatcggggcagccgttgcgagacccgacgcggtggtggttgatatcgatggcgacgggagctttataatgaacgttcaagaattagcgacaatccgcgtcgaaaatctgccggttaagatattattacttaataatcagcatttgggtatggtggttcaatgggaggatcggttttacaaagcgaatcgggcacatacttacttaggaaacccgtcaaacgagactgaaatattcccgaacatgttgaagtttgcggaagcgtgtggtatcccggctgcccgagtgacccggaaggcggacgttcgggcagctattcagaagatgttggatacaccggggccgtacttgttggatgttattgtgccacatcaagaacatgttttgcccatgattccagctggcggcggtttcgcggatgtgatcactgaaggtgatggcagaataaaatactgaggtttgaagt

SEQ ID NO: 24 - DNA, 1981 bp, Coreopsis tinctoria
atggcggctgtcactccctcaagctctcccatctccacctatccctcaccgccgtccgccgccgccaccaccaccacattccgccccaccctccttcctcgtctcacattctcagcctcttctactcccccaaaacaacaccgtctcaccatcgttaacgtcctctccaactcgaaacccaccaccacaaccaccactcattcaccccctccgtttcccgctcggttcgcccctgaccagccccgtaaaggctccgacgtcctcgtagaagccctcgaacgcgaaggcgtcaccgacgtcttcgcctacccgggtggcgcctctatggagatccaccaagccctaactcggtccaaaatcatccgtaacgtcctcccgcgtcacgagcaaggcggcgtcttcgcggccgaggggtacgcccgggcctctggtttacccggcgtgtgcatagccacttccggccccggagctacaaatttggttagcggtcttgcggatgcattacttgacagtgtccccatggtcgcaattacgggtcaagtcccgcggagaatgatcggtaccgatgcgtttcaagaaaccccgattgttgaggtaacacggtctattacaaagcataattaccttgtgttagatgtcgaggatattcctagaattgttagggaggccttttatcttgcgagatcgggtcggcccggcccggttttgattgatatcccgaaagatatacaacaacaattagtggtgccgaaatgggatgaaccgatgaggttaccgggttatcttgctaggttaccgaaaacacaaagtgatgataatagtttagagcagattgttcggttgatatcggaagcgaaaagaccggttttgtatgtgggtggtggttgtttgaattccggtgacgagttggggaggtttgtcgagcttacggggatcccggttgctagtactttaatgggtcttggggcgttcccggcttcgagcgatttatcgcttcatatgcttggaatgcacgggacggtttatgcgaattacgcggtcgataagagtgatttgttgcttgcgttcggggtgcggttcgatgatcgagtgacggggaagctcgaggcgtttgctagccgagctaaaatcgtgcacattgatatcgattcggctgaaatcgggaagaataagcagcctcatgtgtcggtttgtggtgatataaaggtcgcattacaaggtctcaacaagattttggagggaaaaggttcggttactaatctcgattttgcgaattggaggaaggagttggacgagcaaaaggtgaagtttccgttaagttttaaaacgtttggtgaagcgattccgccacaatacgcgattcaggttcttgacgagttaacaggcggtaatgcgattattagcaccggggtcgggcaacatcagatgtgggctgcccagttttacaagtacaacaaaccgagacagtggttgacttcgggtggactcggggcgatgggtttcgggctgcccgctgctatcggggcagctgttgcgagaccggatgcggtggtggtggatatcgatggtgacgggagctttataatgaacgttcaagaattagcgacaatccgcgtcgaaaatctgccggttaagatattattacttaataatcagcatttgggtatggtggttcaatgggaggatcggttttacaaagcgaatcgggctcatacttacttaggaaacccgtcaaacgagactgaaatattcccgaacatgttgaagtttgcggaagcgtgtggtatcccggccgccagagtgacccgaaaggcggacgtccgggcggctattcagaagatgttggatacgccggggccgtacttgttggatgttattgtgccacatcaagaacatgttttgcccatgattccagctggcggcggtttcgcggatgtgatcactgaaggtgatggcagaattaaatactgaggtttgaagt

SEQ ID NO: 25 - protein, 656 aa, Coreopsis tinctoria
MAAVTPSNPPISTYPSPPSAAATTTTNRPTLLPRLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLARSGRPGPVLIDIPKDIQQQLVVPKWDEPMRLPGYLARLPKTQSDDNSLDQIVRLISEAKRPVLYVGGGCLNSGDELGRFVELTGIPVASTLMGLGAFPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 26 - protein, 657 aa, Coreopsis tinctoria
MAAVTDSNPPFSTYPSPPSAAATTTTTFRPTLLPHLTFSASSTLPKRHRLTIVNVLSNSKPTTTTTCHSPPPFPARFAPDQPRKGSDVLIDPLKGQGVPDVFAYPGGASMEIHQALTRSKIIRNVLPRHEQGGVFAAEGYPRASGLPGVCIPTSGPGTTNLVSGRADALLESVRMVAITGQVPPRMIGTDAFQETPIVEVTRSITKHNYLVLDVEDIPRIVREAFYLARSGRPGPVLIDIPKDIQQQLVVPKWDEPMRLPGYLARLPKTQSDDNSLDQIVRLISEAKRPVLYVGGGCLNSGDELGRFVELTGIPVASTLMGLGAFPASSDLSLHMLGMHGTVYANYAVDKSDLLLAFGVRFDDRVTGKLEAFASRAKIVHIDIDSAEIGKNKQPHVSVCGDIKVALQGLNKILEGKDSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 27 - protein, 656 aa, Coreopsis tinctoria
MAAVTPSNPPISTYPSPPSAAATTTTNRPTLLPRLTFSASSTLPKRHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLARSGRPGPVLIDIPKDIQQQLVVPKWDEPMRLPGYLARLPKTQSDDNSLEQIVRLISEAKRPVLYVGGGCLNSGEELGRFVELTGIPVASTLMGLGAYPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY

SEQ ID NO: 28 - protein, 656 aa, Coreopsis tinctoria
MAAVTPSSSPISTYPSPPSAAATTTTFRPTLLPRLTFSASSTPPKQHRLTIVNVLSNSKPTTTTTTHSPPPFPARFAPDQPRKGSDVLVEALEREGVTDVFAYPGGASMEIHQALTRSKIIRNVLPRHEQGGVFAAEGYARASGLPGVCIATSGPGATNLVSGLADALLDSVPMVAITGQVPRRMIGTDAFQETPIVEVTRSITKHNYLVLDVEDIPRIVREAFYLARSGRPGPVLIDIPKDIQQQLVVPKWDEPMRLPGYLARLPKTQSDDNSLEQIVRLISEAKRPVLYVGGGCLNSGDELGRFVELTGIPVASTLMGLGAFPASSDLSLHMLGMHGTVYANYAVDKSDLLLAFGVRFDDRVTGKLEAFASRAKIVHIDIDSAEIGKNKQPHVSVCGDIKVALQGLNKILEGKGSVTNLDFANWRKELDEQKVKFPLSFKTFGEAIPPQYAIQVLDELTGGNAIISTGVGQHQMWAAQFYKYNKPRQWLTSGGLGAMGFGLPAAIGAAVARPDAVVVDIDGDGSFIMNVQELATIRVENLPVKILLLNNQHLGMVVQWEDRFYKANRAHTYLGNPSNETEIFPNMLKFAEACGIPAARVTRKADVRAAIQKMLDTPGPYLLDVIVPHQEHVLPMIPAGGGFADVITEGDGRIKY